SEQUENCE LISTING (4 sequences)

SEQ ID NO: 1 - protein, 135 aa, Artificial Sequence
  MAB heavy chain
MEWSRVFIFLLSVTAGVHSQVQLQQSGAELVRPGTSVKVSCKASGYAFTYYLIEWVKQRPGQGLEWIGVINPGSGGTNYNEKFKGKATLTADKSSSTAYMQLSSLTSDDSAVYFCARNWMNFDYWGQGTTLTVSS

SEQ ID NO: 2 - protein, 132 aa, Artificial Sequence
  MAB light chain
MRCLAEFLGLLVLWIPGAIGDIVMTQAAPSVSVTPGESVSISCRSSKSLLHSNGNTYLYWFLQRPGQSPQFLIYRMSNLASGVPDRFSGSGSGTAFTLRISRVEAEDVGVYYCMQHLEYPYTFGGGTKLEIK

SEQ ID NO: 3 - protein, 116 aa, Artificial Sequence
  Humanized MAB heavy chain
QVQLVQSGAEVKKPGASVKVSCKASGYAFTYYLIEWVRQAPGQGLEWIGVINPGSGGTNYNEKFKGRATITADKSTSTAYMELSSLRSEDTAVYFCARNWMNFDYWGQGTTVTVSS

SEQ ID NO: 4 - protein, 112 aa, Artifical Sequence
  Humanized MAB light chain
DIVMTQTPLSLSVTPGQPASISCRSSKSLLHSNGNTYLYWFLQKPGQSPQFLIYRMSNLASGVPDRFSGSGSGTDFTLKISRVEAEDVGVYYCMQHLEYPYTFGGGTKVEIK

(19) United States
(12) Patent Application Publication
(10) Pub. No.: US 2011/0044981 A1
(43) Pub. Date: Feb. 24, 2011

(54) METHODS AND COMPOSITIONS FOR TREATMENT OF PULMONARY FIBROTIC DISORDERS

(76) Inventors: Rhyannon SPANGLER, Redwood City, CA (US); Victoria SMITH, Burlingame, CA (US)

Correspondence Address: Arresto BioSciences;Bozicevic, Field & Francis LLP, 1900 University Ave, Suite 200, East Palo Alto, CA, 94303, US

(21) Appl. No.: 12/860,834

(22) Filed: Aug. 20, 2010

Related U.S. Application Data
(60) Provisional application No. 61/235,846, filed on Aug. 21, 2009.

(51) Int. Cl. A61K 39/395; G01N 33/566; A61P 11/00

(52) U.S. Cl. 424/133.1; 424/158.1; 435/7.21

(57) ABSTRACT

Disclosed herein are methods and compositions for preventing and treating pulmonary fibrotic disorders, and for reducing or reversing the symptoms of pulmonary fibrotic disorders, such as idiopathic pulmonary fibrosis. The compositions include inhibitors of the LOXL2 protein, and the methods include methods for making and using the inhibitors.

### CROSS REFERENCE TO RELATED APPLICATIONS

This application claims the benefit of U.S. provisional patent application No. 61/235,846 filed Aug. 21, 2009, the disclosure of which in incorporated by reference, in its entirety, for all purposes.

### STATEMENT REGARDING FEDERAL SUPPORT

Not applicable.

### FIELD

The disclosure is in the field of pulmonary fibrotic disorders; for example, idiopathic pulmonary fibrosis (IPF).

### INTRODUCTION

Pulmonary fibrotic disorders are characterized by inflammation and a pathological buildup of connective tissue in the lungs and include such conditions as interstitial pneumonia, acute respiratory distress syndrome (ARDS) and idiopathic pulmonary fibrosis (IPF). These are chronic, progressive diseases for which there is currently no effective therapy.

IPF is characterized by inflammation, and eventually fibrosis, of lung tissue; although these two symptoms can also be dissociated. The cause of IPF is unknown; it may arise either from an autoimmune disorder or as a result of infection. Symptoms of IPF include dyspnea (i.e., shortness of breath) which becomes the major symptom as the disease progresses, and dry cough. Death can result from hypoxemia, right-heart failure, heart attack, lung embolism, stroke or lung infection, all of which can be brought on by the disease.

Pathologically, the early stages of IPF are characterized by inflammation of the alveoli, followed by alveolar fibrosis. This includes fibroblast activation, expansion of fibroblasts and myofibroblasts and abnormal deposition of extracellular matrix in the lung parenchyma. Myofibroblasts associated with IPF may be derived from activated fibroblasts, may be descended from circulating bone marrow-derived progenitor cells, or may result from an “epithelial-to-mesenchymal transition (EMT)” of lung alveolar epithelial cells. Fibrotic scarring of the alveoli reduces the capacity for oxygen transfer, leading to hypoxemia. Hypoxemia, in turn, can lead to pulmonary hypertension, which eventually weakens the right ventricle.

Primary treatment for IPF is pharmaceutical, and most IPF sufferers require treatment throughout their lives. The most common drugs used for treatment of IPF are corticosteroids (e.g., prednisone), penicillamine, and various anti neoplastics (e.g., cyclophosphamide, azathiporene, chlorambucil, vincristine and colchicine). Other treatments include oxygen administration and, in extreme cases, lung transplantation.

Significantly, all treatments for IPF other than lung transplantation fail to reverse the fibrotic damage, but merely prevent further fibrosis. Thus, there is a need for non-invasive treatments for IPF that would not only prevent disease progression, but also reverse existing fibrotic damage.

### SUMMARY

Disclosed herein are methods and compositions for preventing and treating pulmonary fibrotic disorders. Also disclosed are methods and compositions for reversing and/or reducing the symptoms of a pulmonary fibrotic disorder.

The compositions of the disclosure include inhibitors of the lysyl oxidase-related protein-2 (LOXL2), such as, for example, small molecules, nucleic acids and proteins (e.g., antibodies; e.g., an anti-LOXL2 antibody). Pharmaceutical compositions including an inhibitor of LOXL2 (e.g., an anti-LOXL2 antibody), optionally in combination with a pharmaceutically acceptable excipient, are also provided.

Exemplary pulmonary fibrotic disorders include idiopathic pulmonary fibrosis (IPF), interstitial pneumonia and acute respiratory distress syndrome (ARDS).

Symptoms of a pulmonary fibrotic disorder can include, but are not limited to, decreased body weight, increased lung weight, pulmonary fibrosis, pathologic lung architecture (e.g., “honeycomb” lung), increased Ashcroft score, increased pulmonary collagen levels, increased number of CD45⁺/collagen⁺ cells, pneumocyte proliferation and expansion and increased leukocyte number in bronchioalveolar lavage (BAL) fluid. Symptoms can also include, for example, increased pulmonary levels of one or more of the following molecules: LOXL2, α-smooth muscle actin (α-SMA), transforming growth factor β-1 (TGFβ-1), stromal derived factor-1 (SDF-1) (e.g., SDF-1α), endothelin-1 (ET-1) and phosphorylated SMAD2.

The disclosed treatment methods include administering an inhibitor of lysyl oxidase-related protein-2 (LOXL2) to a subject with a pulmonary fibrotic disorder. Exemplary inhibitors include, but are not limited to, antibodies to LOXL2. Exemplary antibodies are the AB0023 and AB0024 antibodies disclosed herein.

Also provided are methods for diagnosing a pulmonary fibrotic disorder in a subject by measuring levels of LOXL2 in a sample of pulmonary tissue from the subject, wherein increased LOXL2 levels are indicative of onset or progression of the pulmonary fibrotic disorder. Levels of LOXL2 can be measured by any method known in the art; for example, contacting a sample with an anti-LOXL2 antibody, detecting formation of a complex between the antibody and the LOXL2 in the sample, and measuring the amount of the complex formed. Additional measurement methods include detecting levels of LOXL2 mRNA. Methods for mRNA detection are well-known in the art.

Also indicative of onset or progression of a pulmonary fibrotic disorder are increases in the levels, in pulmonary tissue, of, for example, α-smooth muscle actin (α-SMA), transforming growth factor β-1 (TGFβ-1), stromal derived factor-1 (e.g., SDF-1α or SDF-1β), endothelin-1 (ET-1) and phosphorylated SMAD2.

In additional embodiments, prognostic methods are provided. Thus, the disclosure includes methods for monitoring a subject's response to a therapy for treating a pulmonary fibrotic disorder in a subject by measuring levels of LOXL2 in a sample of pulmonary tissue from the subject, wherein decreased LOXL2 levels are indicative of amelioration of the pulmonary fibrotic disorder. Levels of LOXL2 can be measured by any method known in the art; for example, contacting a sample with an anti-LOXL2 antibody, detecting formation of a complex between the antibody and the LOXL2 in the sample, and measuring the amount of the complex formed. Additional measurement methods include detecting levels of LOXL2 mRNA. Methods for mRNA detection are well-known in the art.

Also indicative of amelioration of a pulmonary fibrotic disorder are decreases in the levels, in pulmonary tissue of, for example, α-smooth muscle actin (α-SMA), transforming growth factor β-1 (TGFβ-1), stromal derived factor-1 (e.g., SDF-1α or SDF-1β), endothelin-1 (ET-1) and phosphorylated SMAD2.

Accordingly, the present disclosure includes, but is not limited to, the following embodiments:

1. A method for the prevention of a pulmonary fibrotic disorder in a subject, the method comprising administering to the subject an inhibitor of the activity of the lysyl oxidase-related-2 protein (LOXL2).

2. The method of embodiment 1, wherein the pulmonary fibrotic disorder is selected from the group consisting of interstitial pneumonia, acute respiratory distress syndrome (ARDS) and idiopathic pulmonary fibrosis (IPF).

3. The method of embodiment 1, wherein the inhibitor is an antibody to LOXL2.

4. The method of embodiment 3, wherein the antibody comprises heavy chain sequences as set forth in SEQ ID NO:1 and light chain sequences as set forth in SEQ ID NO:2.

5. The method of embodiment 3, wherein the antibody is a humanized antibody.

6. The method of embodiment 5, wherein the antibody comprises heavy chain sequences as set forth in SEQ ID NO:3 and light chain sequences as set forth in SEQ ID NO:4.

7. A method for the treatment of a pulmonary fibrotic disorder in a subject, the method comprising administering to the subject an inhibitor of the activity of the lysyl oxidase-related-2 protein (LOXL2).

8. The method of embodiment 7, wherein the pulmonary fibrotic disorder is selected from the group consisting of interstitial pneumonia, acute respiratory distress syndrome (ARDS) and idiopathic pulmonary fibrosis (IPF).

9. The method of embodiment 7, wherein the inhibitor is an antibody to LOXL2.

10. The method of embodiment 9, wherein the antibody comprises heavy chain sequences as set forth in SEQ ID NO:1 and light chain sequences as set forth in SEQ ID NO:2.

11. The method of embodiment 9, wherein the antibody is a humanized antibody.

12. The method of embodiment 11, wherein the antibody comprises heavy chain sequences as set forth in SEQ ID NO:3 and light chain sequences as set forth in SEQ ID NO:4.

13. A method for reversing the symptoms of a pulmonary fibrotic disorder in a subject, the method comprising administering to the subject an inhibitor of the activity of the lysyl oxidase-related-2 protein (LOXL2).

14. The method of embodiment 13, wherein the pulmonary fibrotic disorder is selected from the group consisting of interstitial pneumonia, acute respiratory distress syndrome (ARDS) and idiopathic pulmonary fibrosis (IPF).

15. The method of embodiment 13, wherein the inhibitor is an antibody to LOXL2.

16. The method of embodiment 15, wherein the antibody comprises heavy chain sequences as set forth in SEQ ID NO:1 and light chain sequences as set forth in SEQ ID NO:2.

17. The method of embodiment 15, wherein the antibody is a humanized antibody.

18. The method of embodiment 17, wherein the antibody comprises heavy chain sequences as set forth in SEQ ID NO:3 and light chain sequences as set forth in SEQ ID NO:4.

19. The method of embodiment 13, wherein the symptom is selected from the group consisting of decreased body weight, increased lung weight, fibrosis, lung architecture, increased Ashcroft score, increased pulmonary collagen levels, and increased number of CD45⁺/collagen⁺ cells.

20. The method of embodiment 13, wherein the symptom is an increased level of one or more molecules selected from the group consisting of LOXL2, α-smooth muscle actin (α-SMA), transforming growth factor β-1 (TGFβ-1), stromal derived factor-1α (SDF-1α), endothelin-1 (ET-1) and phosphorylated SMAD2.

21. The method of embodiment 13, wherein the symptom is increased leukocyte number in bronchioalveolar lavage (BAL) fluid.

22. A pharmaceutical composition for the prevention or treatment of a pulmonary fibrotic disorder, or for reversing the symptoms of a pulmonary fibrotic disorder in a subject, wherein the composition comprises an inhibitor of the activity of the lysyl oxidase-related-2 protein (LOXL2) and a pharmaceutically acceptable excipient.

23. The composition of embodiment 22, wherein the pulmonary fibrotic disorder is selected from the group consisting of interstitial pneumonia, acute respiratory distress syndrome (ARDS) and idiopathic pulmonary fibrosis (IPF).

24. The composition of embodiment 22, wherein the inhibitor is an antibody to LOXL2.

25. The composition of embodiment 24, wherein the antibody comprises heavy chain sequences as set forth in SEQ ID NO:1 and light chain sequences as set forth in SEQ ID NO:2.

26. The composition of embodiment 24, wherein the antibody is a humanized antibody.

27. The composition of embodiment 26, wherein the antibody comprises heavy chain sequences as set forth in SEQ ID NO:3 and light chain sequences as set forth in SEQ ID NO:4.

28. The composition of embodiment 22, wherein the symptom is selected from the group consisting of decreased body weight, increased lung weight, fibrosis, lung architecture, increased Ashcroft score, increased pulmonary collagen levels, and increased number of CD45⁺/collagen⁺ cells.

29. The composition of embodiment 22, wherein the symptom is an increased level of one or more molecules selected from the group consisting of LOXL2, α-smooth muscle actin (α-SMA), transforming growth factor β-1 (TGFβ-1), stromal derived factor-1α (SDF-1α), endothelin-1 (ET-1) and phosphorylated SMAD2.

30. The composition of embodiment 22, wherein the symptom is increased leukocyte number in bronchioalveolar lavage (BAL) fluid.

31. A method for diagnosing a pulmonary fibrotic disorder in a subject, the method comprising:
(a) obtaining a sample of pulmonary tissue from the subject; and
(b) determining the levels of LOXL2 in the sample;

wherein an increased level of LOXL2 in the sample, compared to a control sample, indicates the existence of a pulmonary fibrotic disorder.

32. The method of embodiment 31, wherein the pulmonary fibrotic disorder is selected from the group consisting of interstitial pneumonia, acute respiratory distress syndrome (ARDS) and idiopathic pulmonary fibrosis (IPF).

33. The method of embodiment 31, wherein the levels of LOXL2 in the sample are determined by contacting the sample with an antibody to LOXL2, so as to allow the formation of a complex between the antibody and the LOXL2 in the sample, and measuring the amount of complex that is formed.

34. The method of embodiment 33, wherein the antibody comprises heavy chain sequences as set forth in SEQ ID NO:1 and light chain sequences as set forth in SEQ ID NO:2.

35. The method of embodiment 33, wherein the antibody is a humanized antibody.

36. The method of embodiment 35, wherein the antibody comprises heavy chain sequences as set forth in SEQ ID NO:3 and light chain sequences as set forth in SEQ ID NO:4.

37. A method for monitoring a subject's response to a therapy for treating a pulmonary fibrotic disorder, the method comprising:
(a) obtaining a sample of pulmonary tissue from the subject; and
(b) determining the levels of LOXL2 in the sample;

wherein a decreased level of LOXL2 in the sample, compared to a control sample, indicates an amelioration of the pulmonary fibrotic disorder.

38. The method of embodiment 37, wherein the pulmonary fibrotic disorder is selected from the group consisting of interstitial pneumonia, acute respiratory distress syndrome (ARDS) and idiopathic pulmonary fibrosis (IPF).

39. The method of embodiment 37, wherein the levels of LOXL2 in the sample are determined by contacting the sample with an antibody to LOXL2, so as to allow the formation of a complex between the antibody and the LOXL2 in the sample, and measuring the amount of complex that is formed.

40. The method of embodiment 39, wherein the antibody comprises heavy chain sequences as set forth in SEQ ID NO:1 and light chain sequences as set forth in SEQ ID NO:2.

41. The method of embodiment 39, wherein the antibody is a humanized antibody.

42. The method of embodiment 41, wherein the antibody comprises heavy chain sequences as set forth in SEQ ID NO:3 and light chain sequences as set forth in SEQ ID NO:4.

43. The method of embodiment 37, wherein the treatment comprises administering, to the subject, an inhibitor of LOXL2.

44. The method of embodiment 43, wherein the inhibitor is an antibody.

45. The method of embodiment 44, wherein the inhibitor comprises heavy chain sequences as set forth in SEQ ID NO:1 and light chain sequences as set forth in SEQ ID NO:2.

46. The method of embodiment 44, wherein the inhibitor is a humanized antibody.

47. The method of embodiment 46, wherein the inhibitor comprises heavy chain sequences as set forth in SEQ ID NO:3 and light chain sequences as set forth in SEQ ID NO:4.

48. An inhibitor of the activity of the lysyl oxidase-related-2 protein (LOXL2) for use in the prevention of a pulmonary fibrotic disorder.

49. The inhibitor of embodiment 48, wherein the pulmonary fibrotic disorder is selected from the group consisting of interstitial pneumonia, acute respiratory distress syndrome (ARDS) and idiopathic pulmonary fibrosis (IPF).

50. The inhibitor of embodiment 48, wherein the inhibitor is an antibody to LOXL2.

51. The inhibitor of embodiment 50, wherein the antibody comprises heavy chain sequences as set forth in SEQ ID NO:1 and light chain sequences as set forth in SEQ ID NO:2.

52. The inhibitor of embodiment 50, wherein the antibody is a humanized antibody.

53. The inhibitor of embodiment 52, wherein the antibody comprises heavy chain sequences as set forth in SEQ ID NO:3 and light chain sequences as set forth in SEQ ID NO:4.

54. An inhibitor of the activity of the lysyl oxidase-related-2 protein (LOXL2) for use in the treatment of a pulmonary fibrotic disorder.

55. The inhibitor of embodiment 54, wherein the pulmonary fibrotic disorder is selected from the group consisting of interstitial pneumonia, acute respiratory distress syndrome (ARDS) and idiopathic pulmonary fibrosis (IPF).

56. The inhibitor of embodiment 54, wherein the inhibitor is an antibody to LOXL2.

57. The inhibitor of embodiment 56, wherein the antibody comprises heavy chain sequences as set forth in SEQ ID NO:1 and light chain sequences as set forth in SEQ ID NO:2.

58. The inhibitor of embodiment 56, wherein the antibody is a humanized antibody.

59. The inhibitor of embodiment 58, wherein the antibody comprises heavy chain sequences as set forth in SEQ ID NO:3 and light chain sequences as set forth in SEQ ID NO:4.

60. An inhibitor of the activity of the lysyl oxidase-related-2 protein (LOXL2) for use in reversing the symptoms of a pulmonary fibrotic disorder in a subject.

61. The inhibitor of embodiment 60, wherein the pulmonary fibrotic disorder is selected from the group consisting of interstitial pneumonia, acute respiratory distress syndrome (ARDS) and idiopathic pulmonary fibrosis (IPF).

62. The inhibitor of embodiment 60, wherein the inhibitor is an antibody to LOXL2.

63. The inhibitor of embodiment 62, wherein the antibody comprises heavy chain sequences as set forth in SEQ ID NO:1 and light chain sequences as set forth in SEQ ID NO:2.

64. The inhibitor of embodiment 62, wherein the antibody is a humanized antibody.

65. The inhibitor of embodiment 64, wherein the antibody comprises heavy chain sequences as set forth in SEQ ID NO:3 and light chain sequences as set forth in SEQ ID NO:4.

66. The inhibitor of embodiment 60, wherein the symptom is selected from the group consisting of decreased body weight, increased lung weight, fibrosis, lung architecture, increased Ashcroft score, increased pulmonary collagen levels, and increased number of CD45⁺/collagen⁺ cells.

67. The inhibitor of embodiment 60, wherein the symptom is an increased level of one or more molecules selected from the group consisting of LOXL2, α-smooth muscle actin (α-SMA), transforming growth factor β-1 (TGFβ-1), stromal derived factor-1α (SDF-1α), endothelin-1 (ET-1) and phosphorylated SMAD2.

68. The inhibitor of embodiment 60, wherein the symptom is increased leukocyte number in bronchioalveolar lavage (BAL) fluid.

### BRIEF DESCRIPTION OF THE DRAWINGS

FIG. 1 shows average body weight over the course of the prevention study. Diamonds indicate control animals treated with saline (Group 1); asterisks indicate animals treated with bleomycin on Day 0 (Group 2); and circles indicate animals pretreated with anti-LOXL2 antibody, treated with bleomycin on Day 0, then treated twice weekly with anti-LOXL2 antibody (Group 3).

FIG. 2 shows average leukocyte number in BAL fluid from (left-to-right) saline-treated animals (Group 1), bleomycin-treated animals (Group 2) and bleomycin-treated animals that were pre- and post-treated with anti-LOXL2 (Group 3).

FIG. 3 shows sections of lung analyzed by immunohistochemistry for α-smooth muscle actin (α-SMA, left panels) and LOXL2 (right panels). The upper panels show sections from animals treated with bleomycin and antibody diluent (Group 2); the lower panels show sections from animals treated with bleomycin, and also pre- and post-treated with anti-LOXL2 antibody (AB0023).

FIG. 4 shows average area of LOXL2 signal in sections of lung from animals in Group 1 (“Saline”), Group 2 (“Bleo:Vehicle”) and Group 3 (“Bleo:AB0023”).

FIG. 5 shows average area of α-SMA signal in sections of lung from animals in Group 1 (“Saline”), Group 2 (“Bleo:Vehicle”) and Group 3 (“Bleo:AB0023”).

FIG. 6 shows H&E-stained sections of lungs from animals treated with bleomycin

(Group 2, top left) and bleomycin+anti-LOXL2 antibody (Group 3, top right). Magnification is 20×. Ashcroft scores for lungs from control animals (“Saline control”), bleomycin-treated animals (“Bleomycin: vehicle”) and animals treated with bleomycin and anti-LOXL2 antibody (“Bleomycin: AB0023”) are shown in the bottom panel.

FIG. 7 shows Sirius Red-stained sections of lungs, viewed under transmitted light, from animals treated with bleomycin (Group 2, top left) and bleomycin+anti-LOXL2 antibody (Group 3, top right). Magnification is 20×. Quantitation of levels of cross-linked collagen (as determined by detecting Sirius Red staining under polarized light) for lungs from bleomycin-treated animals (“Bleomycin: vehicle”) and animals treated with bleomycin and anti-LOXL2 antibody (“Bleomycin: AB0023”) is shown in the bottom panel.

FIG. 8 shows sections assayed for the presence of stromal-derived factor-1α (SDF-1α) by immunohistochemistry, in sections of lungs from animals treated with bleomycin (Group 2, top left) and bleomycin+anti-LOXL2 antibody (Group 3, top right). Magnification is 20×. Quantitation of SDF-1α signal in lung sections from control animals (“Saline”), bleomycin-treated animals (“Bleo-vehicle”) and animals treated with bleomycin and anti-LOXL2 antibody (“Bleo-AB0023”) is shown in the bottom panel.

FIG. 9 shows sections assayed for the presence of TGFβ-1 by immunohistochemistry, in sections of lungs from animals treated with bleomycin (Group 2, top left) and bleomycin+anti-LOXL2 antibody (Group 3, top right). Magnification is 20×. Quantitation of TGFβ-1 signal in lung sections from control animals (“Saline”), bleomycin-treated animals (“Bleo-vehicle”) and animals treated with bleomycin and anti-LOXL2 antibody (“Bleo-AB0023”) is shown in the bottom panel.

FIG. 10 shows relative levels of p-SMAD2 in bleomycin-treated mice, that were also treated with either an anti-LOXL2 antibody (AB0023, right) or a control antibody (AC-1, left), determined by ELISA.

FIG. 11 shows sections assayed for the presence of endothelin-1 (ET-1) by immunohistochemistry, in sections of lungs from animals treated with bleomycin (Group 2, top left) and bleomycin+anti-LOXL2 antibody (Group 3, top right). Magnification is 20×. Quantitation of ET-1 signal in lung sections from control animals (“Saline”), bleomycin-treated animals (“Bleo:vehicle”) and animals treated with bleomycin and anti-LOXL2 antibody (“Bleo:AB0023”) is shown in the bottom panel.

FIG. 12 shows representative images of lung sections stained for type I collagen (green) and CD45 (red). Magnification is 20× in the upper panels and 63× in the lower panels. The two left panels show lung sections from animals that had been treated with bleomycin (“1U Bleomycin:Vehicle”) and the two right panels show lung sections from animals that had been treated with bleomycin and anti-LOXL2 antibody (“1U Bleomycin:AB0023”). Co-localization of CD45-positive cells and collagen (indicated by arrows) indicates the presence of possible fibrocytes; i.e., precursors of fibroblasts that contribute to fibrosis in the lung. Treatment with the antibody reduced the incidence of fibrocyte precursor cells in lung tissue.

FIG. 13 shows measurements of the average increase in body weight of bleomycin-treated animals that had received post-treatment injections of either the anti-LOXL2 antibody AB0023 (upper trace) or a control antibody that does not recognize LOXL2 (AC-1, lower trace).

FIG. 14 shows measurements of lung weight in bleomycin-treated and control animals. Shown in the figure are lung weights from control animals that were not treated with bleomycin (“Saline”), from animals shortly after being treated with bleomycin (“Harvest Rx”), from animals 22 days after bleomycin treatment that had received twice-weekly injections of a control antibody (“Bleo:AC-1”) and from animals 22 days after bleomycin treatment that had received twice-weekly injections of an anti-LOXL2 antibody (“Bleo:AB0023”).

FIG. 15 shows hematoxylin and eosin (H&E)-stained sections of mouse lung. The top panel shows a representative section from a Harvest Rx sample, taken 24-48 hours after initiation of antibody treatment on day 7 after bleomycin administration, showing thickening of the lung tissue and widespread lung damage. The middle panel shows a representative lung section from a bleomycin-treated animal that had received injections of the control AC-1 antibody, in which lung damage has progressed. The bottom panel shows a representative lung section from a bleomycin-treated animal that had received injections of the anti-LOXL2 AB0023 antibody, showing reversal of the lung damage caused by bleomycin treatment and normalization of lung architecture.

FIG. 16 shows Ashcroft scores from control animals that were not treated with bleomycin (“Saline”), from animals shortly after being treated with bleomycin (“Harvest Rx”), from animals 22 days after bleomycin treatment that had received twice-weekly injections of a control antibody (“Bleo-AC1”) and from animals 22 days after bleomycin treatment that had received twice-weekly injections of an anti-LOXL2 antibody (“Bleo-AB0023”).

FIG. 17 shows levels of α-SMA in control animals that were not treated with bleomycin (“Saline”), from animals shortly after being treated with bleomycin (“Harvest Rx”), from animals 22 days after bleomycin treatment that had received twice-weekly injections of a control antibody (“Bleo:AC1”) and from animals 22 days after bleomycin treatment that had received twice-weekly injections of an anti-LOXL2 antibody (“Bleo:AB0023”). α-SMA levels were determined by immunohistochemistry and quantitated using MetaMorph Imaging Software (Molecular Devices, Downingtown, Pa.).

FIG. 18 shows levels of LOXL2 in control animals that were not treated with bleomycin (“Saline”), from animals shortly after being treated with bleomycin (“Harvest Rx”), from animals 22 days after bleomycin treatment that had received twice-weekly injections of a control antibody (“Bleo:AC1”) and from animals 22 days after bleomycin treatment that had received twice-weekly injections of an anti-LOXL2 antibody (“Bleo:AB0023”). LOXL2 levels were determined by immunohistochemistry and quantitated using MetaMorph Imaging Software (Molecular Devices, Downingtown, Pa.).

FIG. 19 shows levels of cross-linked collagen, determined by detection of Sirius Red staining under polarized light, in control animals that were not treated with bleomycin (“Saline”), from animals shortly after being treated with bleomycin (“Harvest Rx”), from animals 22 days after bleomycin treatment that had received twice-weekly injections of a control antibody (“Bleo:AC-1”) and from animals 22 days after bleomycin treatment that had received twice-weekly injections of an anti-LOXL2 antibody (“Bleo:AB0023”).

### DETAILED DESCRIPTION

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N.J., 2000; and the series “Methods in Enzymology,” Academic Press, San Diego, Calif.

Pulmonary Fibrotic Disorders

Pulmonary fibrotic disorders are characterized by inflammation and fibrosis of the lung parenchyma. The etiology of these diseases has not been established, and prognosis is generally poor. Currently, pulmonary fibrotic disorders are classified into the following groups, arranged in order of their frequency of occurrence: idiopathic pulmonary fibrosis (IPF), nonspecific interstitial pneumonia (NSIP), respiratory bronchiolitis-associated interstitial lung disease, desquamative interstitial pneumonia, cryptogenic organizing pneumonia, acute interstitial pneumonia, and lymphocytic interstitial pneumonia (LIP). Acute respiratory distress syndrome (ARDS) has also been identified as a pulmonary fibrotic disorder.

Additional pulmonary fibrotic disorders include scleroderma-associated lung fibrosis and fibrotic damage as a sequelae of sarcoidosis.

Symptoms of pulmonary fibrotic disorders include decreased body weight, increased lung weight, presence of activated fibroblasts or fibrocytes, presence of fibrocyte precursor cells (e.g., cells that express both CD45 and collagen), abnormal lung architecture (including alveolar thickening, proliferation and expansion of pneumocytes, and honeycomb lung), increased Ashcroft score (reflecting general lung structure and architecture), increased collagen levels and an increase in the number of leukocytes in bronchioalveolar lavage fluid.

Molecular symptoms of pulmonary fibrosis include increases in the level of one or more of the following proteins: LOXL2, α-smooth muscle actin (α-SMA), transforming growth factor β-1 (TGFβ-1), stromal derived factor-1α (SDF-1α), stromal derived factor-1β (SDF-1β), endothelin-1 (ET-1) and phosphorylated SMAD2.

Involvement of LOXL2 in Pulmonary Fibrotic Disorders

Examination of lung biopsies from patients with pulmonary fibrosis reveals widespread expression of LOXL2 at all histologically-defined stages of IPF. LOXL2 is particularly strongly expressed in disease-associated vasculature and in regions of matrix remodeling and active fibrogenesis. LOXL2 expression is also detected in reactive Type II pneumocytes of fibrotic lung tissue.

Moreover, sites of LOXL2 overexpression are correlated with sites where alpha-smooth muscle actin (α-SMA) is expressed. SMA is a marker of activated fibroblasts, which are a hallmark of fibrotic tissue. Thus, the primary sources of LOXL2 in fibrotic lung tissue appear to be activated fibroblasts (“fibrocytes”) and disease-associated (“reactive”) pneumocytes.

In light of the overexpression of LOXL2 in fibrotic lungs, and the co-localization of LOXL2 overexpression with sites of fibrogenesis and fibroblast activation, the inventors have determined that inhibition of LOXL2 is an effective method for preventing and/or treating pulmonary fibrotic disorders. Moreover, the inventors have determined that inhibition of LOXL2 reverses the symptoms of pulmonary fibrosis, including those mentioned above. Thus, in contrast to other methods, which block, ameliorate or prevent progression of pulmonary fibrosis; the methods and compositions disclosed herein actually promote healing of fibrotic lung tissue and can therefore be used to reverse the course of pulmonary fibrotic disease.

Lysyl Oxidase-Type Enzymes

As used herein, the term “lysyl oxidase-type enzyme” refers to a member of a family of proteins that, inter alia, catalyzes oxidative deamination of ε-amino groups of lysine and hydroxylysine residues, resulting in conversion of peptidyl lysine to peptidyl-α-aminoadipic-67-semialdehyde (allysine) and the release of stoichiometric quantities of ammonia and hydrogen peroxide:

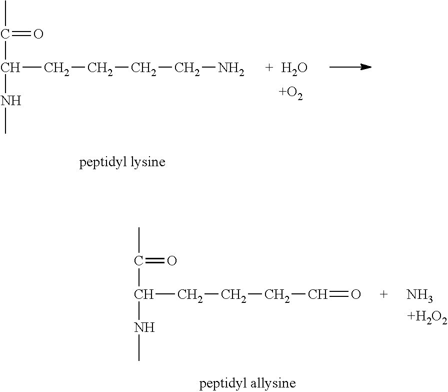

This reaction most often occurs extracellularly, on lysine residues in collagen and elastin. The aldehyde residues of allysine are reactive and can spontaneously condense with other allysine and lysine residues, resulting in crosslinking of collagen molecules to form collagen fibrils.

Lysyl oxidase-type enzymes have been purified from chicken, rat, mouse, bovines and humans. All lysyl oxidase-type enzymes contain a common catalytic domain, approximately 205 amino acids in length, located in the carboxy-terminal portion of the protein and containing the active site of the enzyme. The active site contains a copper-binding site which includes a conserved amino acid sequence containing four histidine residues which coordinate a Cu(II) atom. The active site also contains a lysyltyrosyl quinone (LTQ) cofactor, formed by intramolecular covalent linkage between a lysine and a tyrosine residue (corresponding to lys314 and tyr349 in rat lysyl oxidase, and to lys320 and tyr355 in human lysyl oxidase). The sequence surrounding the tyrosine residue that forms the LTQ cofactor is also conserved among lysyl oxidase-type enzymes. The catalytic domain also contains ten conserved cysteine residues, which participate in the formation of five disulfide bonds. The catalytic domain also includes a fibronectin binding domain. Finally, an amino acid sequence similar to a growth factor and cytokine receptor domain, containing four cysteine residues, is present in the catalytic domain. Despite the presence of these conserved regions, the different lysyl oxidase-type enzymes can be distinguished from one another, both within and outside their catalytic domains, by virtue of regions of divergent nucleotide and amino acid sequence.

The first member of this family of enzymes to be isolated and characterized was lysyl oxidase (EC 1.4.3.13); also known as protein-lysine 6-oxidase, protein-L-lysine:oxygen 6-oxidoreductase (deaminating), or LOX. See, e.g., Harris et al., Biochim. Biophys. Acta 341:332-344 (1974); Rayton et al., J. Biol. Chem. 254:621-626 (1979); Stassen, Biophys. Acta 438:49-60 (1976).

Additional lysyl oxidase-type enzymes were subsequently discovered. These proteins have been dubbed “LOX-like,” or “LOXL.” They all contain the common catalytic domain described above and have similar enzymatic activity. Currently, five different lysyl oxidase-type enzymes are known to exist in both humans and mice: LOX and the four LOX related, or LOX-like proteins LOXL1 (also denoted “lysyl oxidase-like,” “LOXL” or “LOL”), LOXL2 (also denoted “LOR-1”), LOXL3 (also denoted “LOR-2”), and LOXL4. Each of the genes encoding the five different lysyl oxidase-type enzymes resides on a different chromosome. See, for example, Molnar et al., Biochim Biophys Acta. 1647:220-24 (2003); Csiszar, Prog. Nucl. Acid Res. 70:1-32 (2001); WO 01/83702 published on Nov. 8, 2001, and U.S. Pat. No. 6,300,092, all of which are incorporated by reference herein. A LOX-like protein termed LOXC, with some similarity to LOXL4 but with a different expression pattern, has been isolated from a murine EC cell line. Ito et al. (2001) J. Biol. Chem. 276:24023-24029. Two lysyl oxidase-type enzymes, DmLOXL-1 and DmLOXL-2, have been isolated from Drosophila.

Although all lysyl oxidase-type enzymes share a common catalytic domain, they also differ from one another, particularly in their amino-terminal regions. The four LOXL proteins have amino-terminal extensions, compared to LOX. Thus, while human preproLOX (i.e., the primary translation product prior to signal sequence cleavage, see below) contains 417 amino acid residues; LOXL1 contains 574, LOXL2 contains 638, LOXL3 contains 753 and LOXL4 contains 756.

Within their amino-terminal regions, LOXL2, LOXL3 and LOXL4 contain four repeats of the scavenger receptor cysteine-rich (SRCR) domain. These domains are not present in LOX or LOXL1. SRCR domains are found in secreted, transmembrane, or extracellular matrix proteins, and are known to mediate ligand binding in a number of secreted and receptor proteins. Hoheneste et al. (1999) Nat. Struct. Biol. 6:228-232; Sasaki et al. (1998) EMBO J. 17:1606-1613. In addition to its SRCR domains, LOXL3 contains a nuclear localization signal in its amino-terminal region. A proline-rich domain appears to be unique to LOXL1. Molnar et al. (2003) Biochim. Biophys. Acta 1647:220-224. The various lysyl oxidase-type enzymes also differ in their glycosylation patterns.

Tissue distribution also differs among the lysyl oxidase-type enzymes. Human LOX mRNA is highly expressed in the heart, placenta, testis, lung, kidney and uterus, but marginally in the brain and liver. mRNA for human LOXL1 is expressed in the placenta, kidney, muscle, heart, lung, and pancreas and, similar to LOX, is expressed at much lower levels in the brain and liver. Kim et al. (1995) J. Biol. Chem. 270:7176-7182. High levels of LOXL2 mRNA are expressed in the uterus, placenta, and other organs, but as with LOX and LOXL1, low levels are expressed in the brain and liver. Jourdan Le-S aux et al. (1999) J. Biol. Chem. 274:12939:12944. LOXL3 mRNA is highly expressed in the testis, spleen, and prostate, moderately expressed in placenta, and not expressed in the liver, whereas high levels of LOXL4 mRNA are observed in the liver. Huang et al. (2001)Matrix Biol. 20:153-157; Maki and Kivirikko (2001) Biochem. J. 355:381-387; Jourdan Le-Saux et al. (2001)Genomics 74:211-218; Asuncion et al. (2001) Matrix Biol. 20:487-491.

The expression and/or involvement of the different lysyl oxidase-type enzymes in diseases also varies. See, for example, Kagen (1994) Pathol. Res. Pract. 190:910-919; Murawaki et al. (1991) Hepatology 14:1167-1173; Siegel et al. (1978) Proc. Natl. Acad. Sci. USA 75:2945-2949; Jourdan Le-Saux et al. (1994) Biochem. Biophys. Res. Comm. 199:587-592; and Kim et al. (1999) J. Cell Biochem. 72:181-188. Lysyl oxidase-type enzymes have also been implicated in a number of cancers, including head and neck cancer, bladder cancer, colon cancer, esophageal cancer and breast cancer. See, for example, Wu et al. (2007)Cancer Res. 67:4123-4129; Gorough et al. (2007) J. Pathol. 212:74-82; Csiszar (2001) Prog. Nucl. Acid Res. 70:1-32 and Kirschmann et al. (2002) Cancer Res. 62:4478-4483.

Thus, although the lysyl oxidase-type enzymes exhibit some overlap in structure and function, each has distinct structure and functions as well. With respect to structure, for example, certain antibodies raised against the catalytic domain of the human LOX protein do not bind to human LOXL2. With respect to function, it has been reported that targeted deletion of LOX appears to be lethal at parturition in mice, whereas LOXL1 deficiency causes no severe developmental phenotype. Hornstra et al. (2003) J. Biol. Chem. 278:14387-14393; Bronson et al. (2005) Neurosci. Lett. 390:118-122.

Although the most widely documented activity of lysyl oxidase-type enzymes is the oxidation of specific lysine residues in collagen and elastin outside of the cell, there is evidence that lysyl oxidase-type enzymes also participate in a number of intracellular processes. For example, there are reports that some lysyl oxidase-type enzymes regulate gene expression. Li et al. (1997) Proc. Natl. Acad. Sci. USA 94:12817-12822; Giampuzzi et al. (2000) J. Biol. Chem. 275:36341-36349. In addition, LOX has been reported to oxidize lysine residues in histone H1. Additional extracellular activities of LOX include the induction of chemotaxis of monocytes, fibroblasts and smooth muscle cells. Lazarus et al. (1995) Matrix Biol. 14:727-731; Nelson et al. (1988) Proc. Soc. Exp. Biol. Med. 188:346-352. Expression of LOX itself is induced by a number of growth factors and steroids such as TGF-β, TNF-α and interferon. Csiszar (2001) Prog. Nucl. Acid Res. 70:1-32. Recent studies have attributed other roles to LOX in diverse biological functions such as developmental regulation, tumor suppression, cell motility, and cellular senescence.

Examples of lysyl oxidase (LOX) proteins from various sources include enzymes having an amino acid sequence substantially identical to a polypeptide expressed or translated from one of the following sequences: EMBL/GenBank accessions: M94054; AAA59525.1-mRNA; S45875; AAB23549.1-mRNA; 578694; AAB21243.1-mRNA; AF039291; AAD02130.1-mRNA; BC074820; AAH74820.1-mRNA; BC074872; AAH74872.1-mRNA; M84150; AAA59541.1-Genomic DNA. One embodiment of LOX is human lysyl oxidase (hLOX) preproprotein.

Exemplary disclosures of sequences encoding lysyl oxidase-like enzymes are as follows: LOXL1 is encoded by mRNA deposited at GenBank/EMBL BC015090; AAH15090.1; LOXL2 is encoded by mRNA deposited at GenBank/EMBL U89942; LOXL3 is encoded by mRNA deposited at GenBank/EMBL AF282619; AAK51671.1; and LOXL4 is encoded by mRNA deposited at GenBank/EMBL AF338441; AAK71934.1.

The primary translation product of the LOX protein, known as the prepropeptide, contains a signal sequence extending from amino acids 1-21. This signal sequence is released intracellularly by cleavage between Cys21 and Ala22, in both mouse and human LOX, to generate a 46-48 kDa propeptide form of LOX, also referred to herein as the full-length form. The propeptide is N-glycosylated during passage through the Golgi apparatus to yield a 50 kDa protein, then secreted into the extracellular environment. At this stage, the protein is catalytically inactive. A further cleavage, between Gly168 and Asp169 in mouse LOX, and between Gly174 and Asp175 in human LOX, generates the mature, catalytically active, 30-32 kDA enzyme, releasing a 18 kDa propeptide. This final cleavage event is catalyzed by the metalloendoprotease procollagen C-proteinase, also known as bone morphogenetic protein-1 (BMP-1). Interestingly, this enzyme also functions in the processing of LOX's substrate, collagen. The N-glycosyl units are subsequently removed.

Potential signal peptide cleavage sites have been predicted at the amino termini of LOXL1, LOXL2, LOXL3, and LOXL4. The predicted signal cleavage sites are between Gly25 and G1n26 for LOXL1, between Ala25 and G1n26, for LOXL2, between Gly25 and Ser26 for LOXL3 and between Arg23 and Pro24 for LOXL4.

A BMP-1 cleavage site in the LOXL1 protein has been identified between Ser354 and Asp355. Borel et al. (2001) J. Biol. Chem. 276:48944-48949. Potential BMP-1 cleavage sites in other lysyl oxidase-type enzymes have been predicted, based on the consensus sequence for BMP-1 cleavage in procollagens and pro-LOX being at an Ala/Gly-Asp sequence, often followed by an acidic or charged residue. A predicted BMP-1 cleavage site in LOXL3 is located between Gly447 and Asp448; processing at this site may yield a mature peptide of similar size to mature LOX. A potential cleavage site for BMP-1 was also identified within LOXL4, between residues Ala569 and Asp570. Kim et al. (2003) J. Biol. Chem. 278:52071-52074. LOXL2 may also be proteolytically cleaved analogously to the other members of the LOXL family and secreted. Akiri et al. (2003) Cancer Res. 63:1657-1666.

As expected from the existence of a common catalytic domain in the lysyl oxidase-type enzymes, the sequence of the C-terminal 30 kDa region of the proenzyme in which the active site is located is highly conserved (approximately 95%). A more moderate degree of conservation (approximately 60-70%) is observed in the propeptide domain.

For the purposes of the present disclosure, the term “lysyl oxidase-type enzyme” encompasses all five of the lysine oxidizing enzymes discussed above (LOX, LOXL1, LOXL2, LOXL3 and LOXL4), and also encompasses functional fragments and/or derivatives of LOX, LOXL1, LOXL2, LOXL3 and LOXL4 that substantially retain enzymatic activity; e.g., the ability to catalyze deamination of lysyl residues. Typically, a functional fragment or derivative retains at least 50% of its lysine oxidation activity. In some embodiments, a functional fragment or derivative retains at least 60%, at least 70%, at least 80%, at least 90%, at least 95%, at least 99% or 100% of its lysine oxidation activity.

It is also intended that a functional fragment of a lysyl oxidase-type enzyme can include conservative amino acid substitutions (with respect to the native polypeptide sequence) that do not substantially alter catalytic activity. The term “conservative amino acid substitution” refers to grouping of amino acids on the basis of certain common structures and/or properties. With respect to common structures, amino acids can be grouped into those with non-polar side chains (glycine, alanine, valine, leucine, isoleucine, methionine, proline, phenylalanine and tryptophan), those with uncharged polar side chains (serine, threonine, asparagine, glutamine, tyrosine and cysteine) and those with charged polar side chains (lysine, arginine, aspartic acid, glutamic acid and histidine). A group of amino acids containing aromatic side chains includes phenylalanine, tryptophan and tyrosine. Heterocyclic side chains are present in proline, tryptophan and histidine. Within the group of amino acids containing non-polar side chains, those with short hydrocarbon side chains (glycine, alanine, valine, leucine, isoleucine) can be distinguished from those with longer, non-hydrocarbon side chains (methionine, proline, phenylalanine, tryptophan). Within the group of amino acids with charged polar side chains, the acidic amino acids (aspartic acid, glutamic acid) can be distinguished from those with basic side chains (lysine, arginine and histidine).

A functional method for defining common properties of individual amino acids is to analyze the normalized frequencies of amino acid changes between corresponding proteins of homologous organisms (Schulz, G. E. and R. H. Schirmer, Principles of Protein Structure, Springer-Verlag, 1979). According to such analyses, groups of amino acids can be defined in which amino acids within a group are preferentially substituted for one another in homologous proteins, and therefore have similar impact on overall protein structure (Schulz, G. E. and R. H. Schirmer, Principles of Protein Structure, Springer-Verlag, 1979). According to this type of analysis, the following groups of amino acids that can be conservatively substituted for one another can be identified:

(i) amino acids containing a charged group, consisting of Glu, Asp, Lys, Arg and His,

(ii) amino acids containing a positively-charged group, consisting of Lys, Arg and His,

(iii) amino acids containing a negatively-charged group, consisting of Glu and Asp,

(iv) amino acids containing an aromatic group, consisting of Phe, Tyr and Trp,

(v) amino acids containing a nitrogen ring group, consisting of His and Trp,

(vi) amino acids containing a large aliphatic non-polar group, consisting of Val, Leu and Ile,

(vii) amino acids containing a slightly-polar group, consisting of Met and Cys,

(viii) amino acids containing a small-residue group, consisting of Ser, Thr, Asp, Asn, Gly, Ala, Glu, Gln and Pro,

(ix) amino acids containing an aliphatic group consisting of Val, Leu, Be, Met and Cys, and

(x) amino acids containing a hydroxyl group consisting of Ser and Thr.

Thus, as exemplified above, conservative substitutions of amino acids are known to those of skill in this art and can be made generally without altering the biological activity of the resulting molecule. Those of skill in this art also recognize that, in general, single amino acid substitutions in non-essential regions of a polypeptide do not substantially alter biological activity. See, e.g., Watson, et al., “Molecular Biology of the Gene,” 4th Edition, 1987, The Benjamin/Cummings Pub. Co., Menlo Park, Calif., p. 224.

For additional information regarding lysyl oxidase-type enzymes, see, e.g., Rucker et al. (1998) Am. J. Clin. Nutr. 67:996 S-1002S and Kagan et al. (2003) J. Cell. Biochem 88:660-672. See also co-owned United States patent application publication Nos. 2009/0053224 (Feb. 26, 2009) and 2009/0104201 (Apr. 23, 2009); the disclosures of which are incorporated by reference herein.

Modulators of the Activity of Lysyl Oxidase-Type Enzymes

Modulators of the activity of lysyl oxidase-type enzymes include both activators (agonists) and inhibitors (antagonists), and can be selected by using a variety of screening assays. In one embodiment, modulators can be identified by determining if a test compound binds to a lysyl oxidase-type enzyme; wherein, if binding has occurred, the compound is a candidate modulator. Optionally, additional tests can be carried out on such a candidate modulator. Alternatively, a candidate compound can be contacted with a lysyl oxidase-type enzyme, and a biological activity of the lysyl oxidase-type enzyme assayed; a compound that alters the biological activity of the lysyl oxidase-type enzyme is a modulator of a lysyl oxidase-type enzyme. Generally, a compound that reduces a biological activity of a lysyl oxidase-type enzyme is an inhibitor of the enzyme.

Other methods of identifying modulators of the activity of lysyl oxidase-type enzymes include incubating a candidate compound in a cell culture containing one or more lysyl oxidase-type enzymes and assaying one or more biological activities or characteristics of the cells. Compounds that alter the biological activity or characteristic of the cells in the culture are potential modulators of the activity of a lysyl oxidase-type enzyme. Biological activities that can be assayed include, for example, lysine oxidation, peroxide production, ammonia production, levels of lysyl oxidase-type enzyme, levels of mRNA encoding a lysyl oxidase-type enzyme, and/or one or more functions specific to a lysyl oxidase-type enzyme. In additional embodiments of the aforementioned assay, in the absence of contact with the candidate compound, the one or more biological activities or cell characteristics are correlated with levels or activity of one or more lysyl oxidase-type enzymes. For example, the biological activity can be a cellular function such as migration, chemotaxis, epithelial-to-mesenchymal transition, or mesenchymal-to-epithelial transition, and the change is detected by comparison with one or more control or reference sample(s). For example, negative control samples can include a culture with decreased levels of a lysyl oxidase-type enzyme to which the candidate compound is added; or a culture with the same amount of lysyl oxidase-type enzyme as the test culture, but without addition of candidate compound. In some embodiments, separate cultures containing different levels of a lysyl oxidase-type enzyme are contacted with a candidate compound. If a change in biological activity is observed, and if the change is greater in the culture having higher levels of lysyl oxidase-type enzyme, the compound is identified as a modulator of the activity of a lysyl oxidase-type enzyme. Determination of whether the compound is an activator or an inhibitor of a lysyl oxidase-type enzyme may be apparent from the phenotype induced by the compound, or may require further assay, such as a test of the effect of the compound on the enzymatic activity of one or more lysyl oxidase-type enzymes.

Methods for obtaining lysysl oxidase-type enzymes, either biochemically or recombinantly, as well as methods for cell culture and enzymatic assay to identify modulators of the activity of lysyl oxidase-type enzymes as described above, are known in the art.

The enzymatic activity of a lysyl oxidase-type enzyme can be assayed by a number of different methods. For example, lysyl oxidase enzymatic activity can be assessed by detecting and/or quantitating production of hydrogen peroxide, ammonium ion, and/or aldehyde, by assaying lysine oxidation and/or collagen crosslinking, or by measuring cellular invasive capacity, cell adhesion, cell growth or metastatic growth. See, for example, Trackman et al. (1981) Anal. Biochem. 113:336-342; Kagan et al. (1982) Meth. Enzymol. 82A:637-649; Palamakumbura et al. (2002) Anal. Biochem. 300:245-251; Albini et al. (1987) Cancer Res. 47:3239-3245; Kamath et al. (2001) Cancer Res. 61:5933-5940; U.S. Pat. No. 4,997,854 and U.S. patent application publication No. 2004/0248871.

Test compounds include, but are not limited to, small organic compounds (e.g., organic molecules having a molecular weight between about 50 and about 2,500 Da), nucleic acids or proteins, for example. The compound or plurality of compounds can be chemically synthesized or microbiologically produced and/or comprised in, for example, samples, e.g., cell extracts from, e.g., plants, animals or microorganisms. Furthermore, the compound(s) can be known in the art but hitherto not known to be capable of modulating the activity of a lysyl oxidase-type enzyme. The reaction mixture for assaying for a modulator of a lysyl oxidase-type enzyme can be a cell-free extract or can comprise a cell culture or tissue culture. A plurality of compounds can be, e.g., added to a reaction mixture, added to a culture medium, injected into a cell or administered to a transgenic animal. The cell or tissue employed in the assay can be, for example, a bacterial cell, a fungal cell, an insect cell, a vertebrate cell, a mammalian cell, a primate cell, a human cell or can comprise or be obtained from a non-human transgenic animal.

Several methods are known to the person skilled in the art for producing and screening large libraries to identify compounds having specific affinity for a target, such as a lysyl oxidase-type enzyme. These methods include phage display method in which randomized peptides are displayed from phage and screened by affinity chromatography using an immobilized receptor. See, e.g., WO 91/17271, WO 92/01047, and U.S. Pat. No. 5,223,409. In another approach, combinatorial libraries of polymers immobilized on a solid support (e.g., a “chip”) are synthesized using photolithography. See, e.g., U.S. Pat. No. 5,143,854, WO 90/15070 and WO 92/10092. The immobilized polymers are contacted with a labeled receptor (e.g., a lysyl oxidase-type enzyme) and the support is scanned to determine the location of label, to thereby identify polymers binding to the receptor.

The synthesis and screening of peptide libraries on continuous cellulose membrane supports that can be used for identifying binding ligands of a polypeptide of interest (e.g., a lysyl oxidase-type enzyme) is described, for example, in Kramer (1998) Methods Mol. Biol. 87: 25-39. Ligands identified by such an assay are candidate modulators of the protein of interest, and can be selected for further testing. This method can also be used, for example, for determining the binding sites and the recognition motifs in a protein of interest. See, for example Rudiger (1997) EMBO J. 16:1501-1507 and Weiergraber (1996) FEBS Lett. 379:122-126.

WO 98/25146 describes additional methods for screening libraries of complexes for compounds having a desired property, e.g., the capacity to agonize, bind to, or antagonize a polypeptide or its cellular receptor. The complexes in such libraries comprise a compound under test, a tag recording at least one step in synthesis of the compound, and a tether susceptible to modification by a reporter molecule. Modification of the tether is used to signify that a complex contains a compound having a desired property. The tag can be decoded to reveal at least one step in the synthesis of such a compound. Other methods for identifying compounds which interact with a lysyl oxidase-type enzyme are, for example, in vitro screening with a phage display system, filter binding assays, and “real time” measuring of interaction using, for example, the BIAcore apparatus (Pharmacia).

All these methods can be used in accordance with the present disclosure to identify activators/agonists and inhibitors/antagonists of lysyl oxidase-type enzymes or related polypeptides.

Another approach to the synthesis of modulators of lysyl oxidase-type enzymes is to use mimetic analogs of peptides. Mimetic peptide analogues can be generated by, for example, substituting stereoisomers, i.e. D-amino acids, for naturally-occurring amino acids; see e.g., Tsukida (1997) J. Med. Chem. 40:3534-3541. Furthermore, pro-mimetic components can be incorporated into a peptide to reestablish conformational properties that may be lost upon removal of part of the original polypeptide. See, e.g., Nachman (1995) Regul. Pept. 57:359-370.

Another method for constructing peptide mimetics is to incorporate achiral o-amino acid residues into a peptide, resulting in the substitution of amide bonds by polymethylene units of an aliphatic chain. Banerjee (1996) Biopolymers 39:769-777. Superactive peptidomimetic analogues of small peptide hormones in other systems have been described. Zhang (1996) Biochem. Biophys. Res. Commun. 224:327-331.

Peptide mimetics of a modulator of a lysyl oxidase-type enzyme can also be identified by the synthesis of peptide mimetic combinatorial libraries through successive amide alkylation, followed by testing of the resulting compounds, e.g., for their binding and immunological properties. Methods for the generation and use of peptidomimetic combinatorial libraries have been described. See, for example, Ostresh, (1996) Methods in Enzymology 267:220-234 and Dorner (1996) Bioorg. Med. Chem. 4:709-715. Furthermore, a three-dimensional and/or crystallographic structure of one or more lysyl oxidase-type enzymes can be used for the design of peptide mimetic inhibitors of the activity of one or more lysyl oxidase-type enzymes. Rose (1996) Biochemistry 35:12933-12944; Rutenber (1996) Bioorg. Med. Chem. 4:1545-1558.

The structure-based design and synthesis of low-molecular-weight synthetic molecules that mimic the activity of native biological polypeptides is further described in, e.g., Dowd (1998) Nature Biotechnol. 16:190-195; Kieber-Emmons (1997) Current Opinion Biotechnol. 8:435-441; Moore (1997)Proc. West Pharmacol. Soc. 40:115-119; Mathews (1997) Proc. West Pharmacol. Soc. 40:121-125; and Mukhija (1998) European J. Biochem. 254:433-438.

It is also well known to the person skilled in the art that it is possible to design, synthesize and evaluate mimetics of small organic compounds that, for example, can act as a substrate or ligand of a lysyl oxidase-type enzyme. For example, it has been described that D-glucose mimetics of hapalosin exhibited similar efficiency as hapalosin in antagonizing multidrug resistance assistance-associated protein in cytotoxicity. Dinh (1998) J. Med. Chem. 41:981-987.

The structure of the lysyl oxidase-type enzymes can be investigated to guide the selection of modulators such as, for example, small molecules, peptides, peptide mimetics and antibodies. Structural properties of a lysyl oxidase-type enzyme can help to identify natural or synthetic molecules that bind to, or function as a ligand, substrate, binding partner or the receptor of, the lysyl oxidase-type enzyme. See, e.g., Engleman (1997) J. Clin. Invest. 99:2284-2292. For example, folding simulations and computer redesign of structural motifs of lysyl oxidase-type enzymes can be performed using appropriate computer programs. Olszewski (1996) Proteins 25:286-299; Hoffman (1995) Comput. Appl. Biosci. 11:675-679. Computer modeling of protein folding can be used for the conformational and energetic analysis of detailed peptide and protein structure. Monge (1995) J. Mol. Biol. 247:995-1012; Renouf (1995) Adv. Exp. Med. Biol. 376:37-45. Appropriate programs can be used for the identification of sites, on lysyl oxidase-type enzymes, that interact with ligands and binding partners, using computer assisted searches for complementary peptide sequences. Fassina (1994) Immunomethods 5:114-120. Additional systems for the design of protein and peptides are described, for example in Berry (1994) Biochem. Soc. Trans. 22:1033-1036; Wodak (1987), Ann. N.Y. Acad. Sci. 501:1-13; and Pabo (1986) Biochemistry 25:5987-5991. The results obtained from the above-described structural analyses can be used for, e.g., the preparation of organic molecules, peptides and peptide mimetics that function as modulators of the activity of one or more lysyl oxidase-type enzymes.

An inhibitor of a lysyl oxidase-type enzyme can be a competitive inhibitor, an uncompetitive inhibitor, a mixed inhibitor or a non-competitive inhibitor. Competitive inhibitors often bear a structural similarity to substrate, usually bind to the active site, and are more effective at lower substrate concentrations. The apparent K_{M} is increased in the presence of a competitive inhibitor. Uncompetitive inhibitors generally bind to the enzyme-substrate complex or to a site that becomes available after substrate is bound at the active site and may distort the active site. Both the apparent K_{M} and the Vₘₐₓ are decreased in the presence of an uncompetitive inhibitor, and substrate concentration has little or no effect on inhibition. Mixed inhibitors are capable of binding both to free enzyme and to the enzyme-substrate complex and thus affect both substrate binding and catalytic activity. Non-competitive inhibition is a special case of mixed inhibition in which the inhibitor binds enzyme and enzyme-substrate complex with equal avidity, and inhibition is not affected by substrate concentration. Non-competitive inhibitors generally bind to enzyme at a region outside the active site. For additional details on enzyme inhibition see, for example, Voet et al. (2008) supra. For enzymes such as the lysyl oxidase-type enzymes, whose natural substrates (e.g., collagen, elastin) are normally present in vast excess in vivo (compared to the concentration of any inhibitor that can be achieved in vivo), noncompetitive inhibitors are advantageous, since inhibition is independent of substrate concentration.

Antibodies

In certain embodiments, a modulator of a lysyl oxidase-type enzyme is an antibody. In additional embodiments, an antibody is an inhibitor of the activity of a lysyl oxidase-type enzyme.

As used herein, the term “antibody” means an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′ F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or an isolated V_{H} or V_{L} region comprising only three of the six CDRs specific for an antigen) has the ability to recognize and bind antigen, although generally at a lower affinity than does the entire Fᵥ fragment.

The “F_{ab}” fragment also contains, in addition to heavy and light chain variable regions, the constant domain of the light chain and the first constant domain (CH₁) of the heavy chain. Fab fragments were originally observed following papain digestion of an antibody. Fab′ fragments differ from Fab fragments in that F(ab′) fragments contain several additional residues at the carboxy terminus of the heavy chain CH₁ domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain, thereby creating two antigen-binding sites. Diabodies are additionally described, for example, in EP 404,097; WO 93/11161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

In some embodiments, an antibody is a humanized antibody or a human antibody. Humanized antibodies include human immununoglobulins (recipient antibody) in which residues from a complementary determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies can also comprise residues that are found neither in the recipient antibody nor in the imported CDR or framework sequences. In certain embodiments, a humanized antibody comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies can also include immunoglobulin fragments, such as Fv, Fab, Fab′, F(ab′)₂ or other antigen-binding subsequences of antibodies.

The humanized antibody can also comprise at least a portion of an immunoglobulin constant region (Fc), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies are known in the art. Generally, a humanized antibody has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies include chimeric antibodies (U.S. Pat. No. 4,816,567), wherein substantially less than an intact human variable domain has been substituted by the corresponding sequence from a non-human species. In certain embodiments, humanized antibodies are human antibodies in which some CDR residues and optionally some framework region residues are substituted by residues from analogous sites in rodent antibodies (e.g., murine monoclonal antibodies).

Human antibodies can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; and Lonberg et al. (1995) Intern. Rev. Immunol. 13:65-93.

Antibodies can be affinity matured using known selection and/or mutagenesis methods as described above. In some embodiments, affinity matured antibodies have an affinity which is five times or more, ten times or more, twenty times or more, or thirty times or more than that of the starting antibody (generally murine, rabbit, chicken, humanized or human) from which the matured antibody is prepared.

An antibody can also be a bispecific antibody. Bispecific antibodies are monoclonal, and may be human or humanized antibodies that have binding specificities for at least two different antigens. In the present case, the two different binding specificities can be directed to two different lysyl oxidase-type enzymes, or to two different epitopes on a single lysyl oxidase-type enzyme.

An antibody as disclosed herein can also be an immunoconjugate. Such immunoconjugates comprise an antibody (e.g., to a lysyl oxidase-type enzyme) conjugated to a second molecule, such as a reporter An immunoconjugate can also comprise an antibody conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

An antibody that “specifically binds to” or is “specific for” a particular polypeptide or an epitope on a particular polypeptide is one that binds to that particular polypeptide or epitope without substantially binding to any other polypeptide or polypeptide epitope. In some embodiments, an antibody of the present disclosure specifically binds to its target with a dissociation constant (K_{d}) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM; in the form of monoclonal antibody, scFv, Fab, or other form of antibody measured at a temperature of about 4° C., 25° C., 37° C. or 42° C.

In certain embodiments, an antibody of the present disclosure binds to one or more processing sites (e.g., sites of proteolytic cleavage) in a lysyl oxidase-type enzyme, thereby effectively blocking processing of the proenzyme or preproenzyme to the catalytically active enzyme, thereby reducing the activity of the lysyl oxidase-type enzyme.

In certain embodiments, an antibody according to the present disclosure binds to human LOXL2 with a greater binding affinity, for example, at least 10 times, at least 100 times, or even at least 1000 times greater than its binding affinity to other lysyl oxidase-type enzymes, e.g., LOX, LOXL1, LOXL3, and LOXL4.

In certain embodiments, an antibody according to the present disclosure is a non-competitive inhibitor of the catalytic activity of a lysyl oxidase-type enzyme. In certain embodiments, an antibody according to the present disclosure binds outside the catalytic domain of a lysyl oxidase-type enzyme. In certain embodiments, an antibody according to the present disclosure binds to the SRCR4 domain of LOXL2. In certain embodiments, an anti-LOXL2 antibody that binds to the SRCR4 domain of LOXL2 and functions as a non-competitive inhibitor is the AB0023 antibody, described in co-owned U.S. Patent Application Publications No. US 2009/0053224 and US 2009/0104201. In certain embodiments, an anti-LOXL2 antibody that binds to the SRCR4 domain of LOXL2 and functions as a non-competitive inhibitor is the AB0024 antibody (a human version of the AB0023 antibody), described in co-owned U.S. Patent Application Publications No. US 2009/0053224 and US 2009/0104201.

Optionally, an antibody according to the present disclosure not only binds to a lysyl oxidase-type enzyme but also reduces or inhibits uptake or internalization of the lysyl oxidase-type enzyme, e.g., via integrin beta 1 or other cellular receptors or proteins. Such an antibody could, for example, bind to extracellular matrix proteins, cellular receptors, and/or integrins.

Exemplary antibodies that recognize lysyl oxidase-type enzymes, and additional disclosure relating to antibodies to lysyl oxidase-type enzymes, is provided in co-owned U.S. Patent Application Publications No. US 2009/0053224 and US 2009/0104201, the disclosures of which are incorporated by reference for the purposes of describing antibodies to lysyl oxidase-type enzymes, their manufacture, and their use.

Polynucleotides for Modulating Expression of Lysyl Oxidase-Type Enzymes

Antisense

Modulation (e.g., inhibition) of a lysyl oxidase-type enzyme can be effected by down-regulating expression of the lysyl oxidase enzyme at either the transcriptional or translational level. One such method of modulation involves the use of antisense oligo- or polynucleotides capable of sequence-specific binding with a mRNA transcript encoding a lysyl oxidase-type enzyme.

Binding of an antisense oligonucleotide (or antisense oligonucleotide analogue) to a target mRNA molecule can lead to the enzymatic cleavage of the hybrid by intracellular RNase H. In certain cases, formation of an antisense RNA-mRNA hybrid can interfere with correct splicing. In both cases, the number of intact, functional target mRNAs, suitable for translation, is reduced or eliminated. In other cases, binding of an antisense oligonucleotide or oligonucleotide analogue to a target mRNA can prevent (e.g., by steric hindrance) ribosome binding, thereby preventing translation of the mRNA.

Antisense oligonucleotides can comprise any type of nucleotide subunit, e.g., they can be DNA, RNA, analogues such as peptide nucleic acids (PNA), or mixtures of the preceding. RNA oligonucleotides form a more stable duplex with a target mRNA molecule, but the unhybridized oligonucleotides are less stable intracellularly than other types of oligonucleotides and oligonucleotide analogues. This can be counteracted by expressing RNA oligonucleotides inside a cell using vectors designed for this purpose. This approach may be used, for example, when attempting to target a mRNA that encodes an abundant and long-lived protein.

Additional considerations can be taken into account when designing antisense oligonucleotides, including: (i) sufficient specificity in binding to the target sequence; (ii) solubility; (iii) stability against intra- and extracellular nucleases; (iv) ability to penetrate the cell membrane; and (v) when used to treat an organism, low toxicity.

Algorithms for identifying oligonucleotide sequences with the highest predicted binding affinity for their target mRNA, based on a thermodynamic cycle that accounts for the energy of structural alterations in both the target mRNA and the oligonucleotide, are available. For example, Walton et al. (1999) Biotechnol. Bioeng. 65:1-9 used such a method to design antisense oligonucleotides directed to rabbit β-globin (RBG) and mouse tumor necrosis factor-α (TNF α) transcripts. The same research group has also reported that the antisense activity of rationally selected oligonucleotides against three model target mRNAs (human lactate dehydrogenase A and B and rat gp130) in cell culture proved effective in almost all cases. This included tests against three different targets in two cell types using oligonucleotides made by both phosphodiester and phosphorothioate chemistries.

In addition, several approaches for designing and predicting efficiency of specific oligonucleotides using an in vitro system are available. See, e.g., Matveeva et al. (1998) Nature Biotechnology 16:1374-1375.

An antisense oligonucleotide according to the present disclosure includes a polynucleotide or a polynucleotide analogue of at least 10 nucleotides, for example, between 10 and 15, between 15 and 20, at least 17, at least 18, at least 19, at least 20, at least 22, at least 25, at least 30, or even at least 40 nucleotides. Such a polynucleotide or polynucleotide analogue is able to anneal or hybridize (i.e., form a double-stranded structure on the basis of base complementarity) in vivo, under physiological conditions, with a mRNA encoding a lysyl oxidase-type enzyme, e.g., LOX or LOXL2.

Antisense oligonucleotides according to the present disclosure can be expressed from a nucleic acid construct administered to a cell or tissue. Optionally, expression of the antisense sequences is controlled by an inducible promoter, such that expression of antisense sequences can be switched on and off in a cell or tissue. Alternatively antisense oligonucleotides can be chemically synthesized and administered directly to a cell or tissue, as part of, for example, a pharmaceutical composition.

Antisense technology has led to the generation of highly accurate antisense design algorithms and a wide variety of oligonucleotide delivery systems, thereby enabling those of ordinary skill in the art to design and implement antisense approaches suitable for downregulating expression of known sequences. For additional information relating to antisense technology, see, for example, Lichtenstein et al., “Antisense Technology: A Practical Approach,” Oxford University Press, 1998.

Small RNA and RNAi

Another method for inhibition of the activity of a lysyl oxidase-type enzyme is RNA interference (RNAi), an approach which utilizes double-stranded small interfering RNA (siRNA) molecules that are homologous to a target mRNA and lead to its degradation. Carthew (2001) Curr. Opin. Cell. Biol. 13:244-248.

RNA interference is typically a two-step process. In the first step, which is termed as the initiation step, input dsRNA is digested into 21-23 nucleotide (nt) small interfering RNAs (siRNAs), probably by the action of Dicer, a member of the RNase III family of double-strand-specific ribonucleases, which cleaves double-stranded RNA in an ATP-dependent manner. Input RNA can be delivered, e.g., directly or via a transgene or a virus. Successive cleavage events degrade the RNA to 19-21 by duplexes (siRNA), each with 2-nucleotide 3′ overhangs. Hutvagner et al. (2002) Curr. Opin. Genet. Dev. 12:225-232; Bernstein (2001) Nature 409:363-366.

In the second, effector step, siRNA duplexes bind to a nuclease complex to form the RNA-induced silencing complex (RISC). An ATP-dependent unwinding of the siRNA duplex is required for activation of the RISC. The active RISC (containing a single siRNA and an RNase) then targets the homologous transcript by base pairing interactions and typically cleaves the mRNA into fragments of approximately 12 nucleotides, starting from the 3′ terminus of the siRNA. Hutvagner et al., supra; Hammond et al. (2001) Nat. Rev. Gen. 2:110-119; Sharp (2001) Genes. Dev. 15:485-490.

RNAi and associated methods are also described in Tuschl (2001) Chem. Biochem. 2:239-245; Cullen (2002) Nat. Immunol. 3:597-599; and Brantl (2002) Biochem. Biophys. Acta. 1575:15-25.

An exemplary strategy for synthesis of RNAi molecules suitable for use with the present disclosure, as inhibitors of the activity of a lysyl oxidase-type enzyme, is to scan the appropriate mRNA sequence downstream of the start codon for AA dinucleotide sequences. Each AA, plus the downstream (i.e., 3′ adjacent) 19 nucleotides, is recorded as a potential siRNA target site. Target sites in coding regions are preferred, since proteins that bind in untranslated regions (UTRs) of a mRNA, and/or translation initiation complexes, may interfere with binding of the siRNA endonuclease complex. Tuschl (2001) supra. It will be appreciated though, that siRNAs directed at untranslated regions can also be effective, as has been demonstrated in the case wherein siRNA directed at the 5′ UTR of the GAPDH gene mediated about 90% decrease in cellular GAPDH mRNA and completely abolished protein level (www.ambion.com/techlib/tn/91/912.html). Once a set of potential target sites is obtained, as described above, the sequences of the potential targets are compared to an appropriate genomic database (e.g., human, mouse, rat etc.) using a sequence alignment software, (such as the BLAST software available from NCBI at www.ncbi.nlm.nih.gov/BLAST/). Potential target sites that exhibit significant homology to other coding sequences are rejected.

Qualifying target sequences are selected as templates for siRNA synthesis. Selected sequences can include those with low G/C content as these have been shown to be more effective in mediating gene silencing, compared to those with G/C content higher than 55%. Several target sites can be selected along the length of the target gene for evaluation. For better evaluation of the selected siRNAs, a negative control is used in conjunction. Negative control siRNA can include a sequence with the same nucleotide composition as a test siRNA, but lacking significant homology to the genome. Thus, for example, a scrambled nucleotide sequence of the siRNA may be used, provided it does not display any significant homology to any other gene.

The siRNA molecules of the present disclosure can be transcribed from expression vectors which can facilitate stable expression of the siRNA transcripts once introduced into a host cell. These vectors are engineered to express small hairpin RNAs (shRNAs), which are processed in vivo into siRNA molecules capable of carrying out gene-specific silencing. See, for example, Brummelkamp et al. (2002) Science 296:550-553; Paddison et al (2002) Genes Dev. 16:948-958; Paul et al. (2002) Nature Biotech. 20:505-508; Yu et al. (2002) Proc. Natl. Acad. Sci. USA 99:6047-6052.

Small hairpin RNAs (shRNAs) are single-stranded polynucleotides that form a double-stranded, hairpin loop structure. The double-stranded region is formed from a first sequence that is hybridizable to a target sequence, such as a polynucleotide encoding a lysyl oxidase-type enzyme (e.g., a LOX or LOXL2 mRNA) and a second sequence that is complementary to the first sequence. The first and second sequences form a double stranded region; while the un-base-paired linker nucleotides that lie between the first and second sequences form a hairpin loop structure. The double-stranded region (stem) of the shRNA can comprise a restriction endonuclease recognition site.

A shRNA molecule can have optional nucleotide overhangs, such as 2-bp overhangs, for example, 3′ UU-overhangs. While there may be variation, stem length typically ranges from approximately 15 to 49, approximately 15 to 35, approximately 19 to 35, approximately 21 to 31 bp, or approximately 21 to 29 bp, and the size of the loop can range from approximately 4 to 30 bp, for example, about 4 to 23 bp.

For expression of shRNAs within cells, plasmid vectors can be employed that contain a promoter (e.g., the RNA Polymerase III H1-RNA promoter or the U6 RNA promoter), a cloning site for insertion of sequences encoding the shRNA, and a transcription termination signal (e.g., a stretch of 4-5 adenine-thymidine base pairs). Polymerase III promoters generally have well-defined transcriptional initiation and termination sites, and their transcripts lack poly(A) tails. The termination signal for these promoters is defined by the polythymidine tract, and the transcript is typically cleaved after the second encoded uridine. Cleavage at this position generates a 3′ UU overhang in the expressed shRNA, which is similar to the 3′ overhangs of synthetic siRNAs. Additional methods for expressing shRNA in mammalian cells are described in the references cited above.

An example of a suitable shRNA expression vector is pSUPER™ (Oligoengine, Inc., Seattle, Wash.), which includes the polymerase-III H1-RNA gene promoter with a well defined transcriptional startsite and a termination signal consisting of five consecutive adenine-thymidine pairs. Brummelkamp et al., supra. The transcription product is cleaved at a site following the second uridine (of the five encoded by the termination sequence), yielding a transcript which resembles the ends of synthetic siRNAs, which also contain nucleotide overhangs. Sequences to be transcribed into shRNA are cloned into such a vector such that they will generate a transcript comprising a first sequence complementary to a portion of a mRNA target (e.g., a mRNA encoding a lysyl oxidase-type enzyme), separated by a short spacer from a second sequence comprising the reverse complement of the first sequence. The resulting transcript folds back on itself to form a stem-loop structure, which mediates RNA interference (RNAi).

Another suitable siRNA expression vector encodes sense and antisense siRNA under the regulation of separate pol III promoters. Miyagishi et al. (2002) Nature Biotech. 20:497-500. The siRNA generated by this vector also includes a five thymidine (T5) termination signal.

siRNAs, shRNAs and/or vectors encoding them can be introduced into cells by a variety of methods, e.g., lipofection. Vector-mediated methods have also been developed. For example, siRNA molecules can be delivered into cells using retroviruses. Delivery of siRNA using retroviruses can provide advantages in certain situations, since retroviral delivery can be efficient, uniform and immediately selects for stable “knock-down” cells. Devroe et al. (2002) BMC Biotechnol. 2:15.

Recent scientific publications have validated the efficacy of such short double stranded RNA molecules in inhibiting target mRNA expression and thus have clearly demonstrated the therapeutic potential of such molecules. For example, RNAi has been utilized for inhibition in cells infected with hepatitis C virus (McCaffrey et al. (2002) Nature 418:38-39), HIV-1 infected cells (Jacque et al. (2002) Nature 418:435-438), cervical cancer cells (Jiang et al. (2002) Oncogene 21:6041-6048) and leukemic cells (Wilda et al. (2002) Oncogene 21:5716-5724).

Methods for Modulating Expression of Lysyl Oxidase-Type Enzymes

Another method for modulating the activity of a lysyl oxidase-type enzyme is to modulate the expression of its encoding gene, leading to lower levels of activity if gene expression is repressed, and higher levels if gene expression is activated. Modulation of gene expression in a cell can be achieved by a number of methods.

For example, oligonucleotides that bind genomic DNA (e.g., regulatory regions of a lysyl oxidase-type gene) by strand displacement or by triple-helix formation can block transcription, thereby preventing expression of a lysyl oxidase-type enzyme. In this regard, the use of so-called “switch back” chemical linking, in which an oligonucleotide recognizes a polypurine stretch on one strand on one strand of its target and a homopurine sequence on the other strand, has been described. Triple-helix formation can also be obtained using oligonucleotides containing artificial bases, thereby extending binding conditions with regard to ionic strength and pH.

Modulation of transcription of a gene encoding a lysyl oxidase-type enzyme can also be achieved, for example, by introducing into cell a fusion protein comprising a functional domain and a DNA-binding domain, or a nucleic acid encoding such a fusion protein. A functional domain can be, for example, a transcriptional activation domain or a transcriptional repression domain. Exemplary transcriptional activation domains include VP16, VP64 and the p65 subunit of NF-κB; exemplary transcriptional repression domains include KRAB, KOX and v-erbA.

In certain embodiments, the DNA-binding domain portion of such a fusion protein is a sequence-specific DNA-binding domain that binds in or near a gene encoding a lysyl oxidase-type enzyme, or in a regulatory region of such a gene. The DNA-binding domain can either naturally bind to a sequence at or near the gene or regulatory region, or can be engineered to so bind. For example, the DNA-binding domain can be obtained from a naturally-occurring protein that regulates expression of a gene encoding a lysyl oxidase-type enzyme. Alternatively, the DNA-binding domain can be engineered to bind to a sequence of choice in or near a gene encoding a lysyl oxidase-type enzyme or in a regulatory region of such a gene.

In this regard, the zinc finger DNA-binding domain is useful, inasmuch as it is possible to engineer zinc finger proteins to bind to any DNA sequence of choice. A zinc finger binding domain comprises one or more zinc finger structures. Miller et al. (1985) EMBO J. 4:1609-1614; Rhodes (1993) Scientific American, February: 56-65; U.S. Pat. No. 6,453,242. Typically, a single zinc finger is about 30 amino acids in length and contains four zinc-coordinating amino acid residues. Structural studies have demonstrated that the canonical (C₂H₂) zinc finger motif contains two beta sheets (held in a beta turn which generally contains two zinc-coordinating cysteine residues) packed against an alpha helix (generally containing two zinc coordinating histidine residues).

Zinc fingers include both canonical C₂H₂ zinc fingers (i.e., those in which the zinc ion is coordinated by two cysteine and two histidine residues) and non-canonical zinc fingers such as, for example, C₃H zinc fingers (those in which the zinc ion is coordinated by three cysteine residues and one histidine residue) and C₄ zinc fingers (those in which the zinc ion is coordinated by four cysteine residues). Non-canonical zinc fingers can also include those in which an amino acid other than cysteine or histidine is substituted for one of these zinc-coordinating residues. See e.g., WO 02/057293 (Jul. 25, 2002) and US 2003/0108880 (Jun. 12, 2003).

Zinc finger binding domains can be engineered to have a novel binding specificity, compared to a naturally-occurring zinc finger protein; thereby allowing the construction of zinc finger binding domains engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. Engineering methods include, but are not limited to, rational design and various types of empirical selection methods.

Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,030,215; 7,067,617; U.S. Patent Application Publication Nos. 2002/0165356; 2004/0197892; 2007/0154989; 2007/0213269; and International Patent Application Publication Nos. WO 98/53059 and WO 2003/016496.

Exemplary selection methods, including phage display, interaction trap, hybrid selection and two-hybrid systems, are disclosed in U.S. Pat. Nos. 5,789,538; 5,925,523; 6,007,988; 6,013,453; 6,140,466; 6,200,759; 6,242,568; 6,410,248; 6,733,970; 6,790,941; 7,029,847 and 7,297,491; as well as U.S. Patent Application Publication Nos. 2007/0009948 and 2007/0009962; WO 98/37186; WO 01/60970 and GB 2,338,237.

Enhancement of binding specificity for zinc finger binding domains has been described, for example, in U.S. Pat. No. 6,794,136 (Sep. 21, 2004). Additional aspects of zinc finger engineering, with respect to inter-finger linker sequences, are disclosed in U.S. Pat. No. 6,479,626 and U.S. Patent Application Publication No. 2003/0119023. See also Moore et al. (2001a) Proc. Natl. Acad. Sci. USA 98:1432-1436; Moore et al. (2001b) Proc. Natl. Acad. Sci. USA 98:1437-1441 and WO 01/53480.

Further details on the use of fusion proteins comprising engineered zinc finger DNA-binding domains are found, for example, in U.S. Pat. Nos. 6,534,261; 6,607,882; 6,824,978; 6,933,113; 6,979,539; 7,013,219; 7,070,934; 7,163,824 and 7,220,719.

Additional methods for modulating the expression of a lysyl oxidase-type enzyme include targeted mutagenesis, either of the gene or of a regulatory region that controls expression of the gene. Exemplary methods for targeted mutagenesis using fusion proteins comprising a nuclease domain and an engineered DNA-binding domain are provided, for example, in U.S. Patent application publications 2005/0064474; 2007/0134796; and 2007/0218528.

Formulations, Kits and Routes of Administration

Therapeutic compositions comprising compounds identified as modulators of the activity of a lysyl oxidase-type enzyme (e.g., inhibitors or activators of a lysyl oxidase-type enzyme) are also provided. Such compositions typically comprise the modulator and a pharmaceutically acceptable carrier. Supplementary active compounds can also be incorporated into the compositions. For example, inhibitors of LOXL2 are useful in combination with a steroid, antibiotic or anti-neoplastic for treatment of a pulmonary fibrotic disorder. Accordingly, therapeutic compositions as disclosed herein can contain both a modulator of the activity of a lysyl oxidase-type enzyme and a steroid, an antibiotic and/or an anti-neoplastic agent.

As used herein, the term “therapeutically effective amount” or “effective amount” refers to an amount of a therapeutic agent that when administered alone or in combination with another therapeutic agent to a cell, tissue, or subject (e.g., a mammal such as a human or a non-human animal such as a primate, rodent, cow, horse, pig, sheep, etc.) is effective to prevent or ameliorate the disease condition or the progression of the disease or to reverse progression of the disease. A therapeutically effective dose further refers to that amount of the compound sufficient to result in full or partial amelioration of symptoms, e.g., treatment, healing, prevention or amelioration of the relevant medical condition, or an increase in rate of treatment, healing, prevention or amelioration of such conditions. A therapeutically effective amount of, for example, an inhibitor of the activity of a lysyl oxidase-type enzyme varies with the type of disease or disorder, extensiveness of the disease or disorder, and size of the organism suffering from the disease or disorder.

The therapeutic compositions disclosed herein are useful for, inter alia, reducing fibrotic damage and reversing the progression of a pulmonary fibrotic disorder. Accordingly, a “therapeutically effective amount” of a modulator (e.g., inhibitor) of the activity of a lysyl oxidase-type enzyme (e.g., LOXL2) can be an amount that results in reversal of pulmonary fibrotic damage. For example, when the LOXL2 inhibitor is an antibody and the antibody is administered in vivo, normal dosage amounts can vary from about 10 ng/kg to up to 100 mg/kg of mammal body weight or more per day, for example, about 1 μg/kg/day to 50 mg/kg/day, optionally about 100 μg/kg/day to 20 mg/kg/day, 500 μg/kg/day to 10 mg/kg/day, or 1 mg/kg/day to 10 mg/kg/day, or about 15 mg/kg/day depending upon, e.g., body weight, route of administration, severity of disease, etc. Dosage amounts can also be administered rather than daily on a schedule of once, twice, or three times per week in an amount of from about 10 ng/kg to up to 100 mg/kg of mammal body weight or more per dose, for example, about 1 μg/kg/dose to 50 mg/kg/dose, optionally about 100 μg/kg/dose to 20 mg/kg/dose, 500 μg/kg/dose to 10 mg/kg/dose, or 1 mg/kg/dose to 10 mg/kg/dose, or about 15 mg/kg/dose. In one example, the dose is about 15/mg/kg administered twice weekly. The periods of treatment can range from, for example, 2 weeks, 3 weeks, 4 weeks, 5 weeks, 6 weeks, or more.

When a modulator of the activity of a lysyl oxidase-type enzyme is used in combination with a steroid, an antibiotic or an anti-neoplastic agent, one can also refer to the therapeutically effective dose of the combination, which is the combined amounts of the modulator and the other agent that result in reduction of pulmonary fibrotic damage, whether administered in combination, serially or simultaneously. More than one combination of concentrations can be therapeutically effective.

Various pharmaceutical compositions and techniques for their preparation and use are known to those of skill in the art in light of the present disclosure. For a detailed listing of suitable pharmacological compositions and techniques for their administration one may refer to the detailed teachings herein, which may be further supplemented by texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

The disclosed therapeutic compositions further include pharmaceutically acceptable materials, compositions or vehicle, such as a liquid or solid filler, diluent, excipient, solvent or encapsulating material, i.e., carriers. These carriers are involved in transporting the subject modulator from one organ, or region of the body, to another organ, or region of the body. Each carrier should be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Another aspect of the present disclosure relates to kits for carrying out the administration of a modulator of the activity of a lysyl oxidase-type enzyme, e.g., a LOXL2 inhibitor. Another aspect of the present disclosure relates to kits for carrying out the combined administration of a modulator of the activity of a lysyl oxidase-type enzyme and a steroid, antibiotic or anti-neoplastic agent. In one embodiment, a kit comprises an inhibitor of the activity of a lysyl oxidase-type enzyme (e.g. an inhibitor of LOXL2) formulated in a pharmaceutical carrier, optionally containing at least one steroid, antibiotic or anti-neoplastic agent, formulated as appropriate, in one or more separate pharmaceutical preparations.

The formulation and delivery methods can be adapted according to the site(s) and degree of fibrotic damage. Exemplary formulations include, but are not limited to, those suitable for parenteral administration, e.g., intrapulmonary, intravenous, intra-arterial, intra-ocular, or subcutaneous administration, including formulations encapsulated in micelles, liposomes or drug-release capsules (active agents incorporated within a biocompatible coating designed for slow-release); ingestible formulations; formulations for topical use, such as eye drops, creams, ointments and gels; and other formulations such as inhalants, aerosols and sprays. The dosage of the compounds of the disclosure will vary according to the extent and severity of the need for treatment, the activity of the administered composition, the general health of the subject, and other considerations well known to the skilled artisan.

In additional embodiments, the compositions described herein are delivered locally, e.g., intrapulmonarily. Thus, a formulation comprising an inhibitor of LOXL2 can be administered by inhalation, and nebulized formulations can be administered either orally or nasally. Localized delivery allows for the delivery of the composition non-systemically, thereby reducing the body burden of the composition as compared to systemic delivery. Such local delivery can be achieved, for example, through the use of various medically implanted devices including, but not limited to, stents and catheters, or can be achieved by inhalation, injection or surgery. Methods for coating, implanting, embedding, and otherwise attaching desired agents to medical devices such as stents and catheters are established in the art and contemplated herein.

Anti-LOXL2 Antibodies

A monoclonal antibody directed against LOXL2 has been described in co-owned United States Patent Application Publication No. US 2009/0053224 (Feb. 26, 2009). This antibody is designated AB0023. Antibodies having a heavy chain having the CDRs (CDR1, CDR2, and CDR3) of AB0023 and having a light chain having the CDRs (CDR1, CDR2, and CDR3) of AB0023 are of interest. The sequence of the CDRs and intervening framework regions of the variable region of its heavy chain is as follows (the sequences of CDR1, CDR2, and CDR3 are underlined):

(SEQ ID NO: 1)
MEWSRVFIFLLSVTAGVHSQVQLQQSGAELVRPGTSVKVSCKASGYAF
TYYLIEWVKQRPGQGLEWIGVINPGSGGTNYNEKFKGKATLTADKSSS
TAYMQLSSLTSDDSAVYFCARNWMNFDYWGQGTTLTVSS

Additional heavy chain variable region amino acid sequences having 75% or more, 80% or more, 90% or more, 95% or more, or 99% or more homology to SEQ ID NO:1 are also provided.

The sequence of the CDRs and intervening framework regions of the variable region of the light chain of the AB0023 antibody is (the sequences of CDR1, CDR2, and CDR3 are underlined):

(SEQ ID NO: 2)
MRCLAEFLGLLVLWIPGAIGDIVMTQAAPSVSVTPGESVSISCRSSKS
LLHSNGNTYLYWFLQRPGQSPQFLIYRMSNLASGVPDRFSGSGSGTAF
TLRISRVEAEDVGVYYCMQHLEYPYTFGGGTKLEIK

Additional light chain variable region amino acid sequences having 75% or more, 80% or more, 90% or more, 95% or more, or 99% or more homology to SEQ ID NO:2 are also provided.

Humanized versions of the above-mentioned anti-LOXL2 monoclonal antibody have been described in co-owned United States Patent Application Publication No. US 2009/0053224 (Feb. 26, 2009). An exemplary humanized antibody is designated AB0024. Humanized antibodies having a heavy chain having the CDRs (CDR1, CDR2, and CDR3) of AB0024 and having a light chain having the CDRs (CDR1, CDR2, and CDR3) of AB0024 are of interest. The sequence of the CDRs and intervening framework regions of the variable region of its heavy chain is as follows (the sequences of CDR1, CDR2, and CDR3 are underlined):

(SEQ ID NO: 3)
QVQLVQSGAEVKKPGASVKVSCKASGYAFTYYLIEWVRQAPGQGLEWI
GVINPGSGGTNYNEKFKGRATITADKSTSTAYMELSSLRSEDTAVYFC
ARNWMNFDYWGQGTTVTVSS

Additional heavy chain variable region amino acid sequences having 75% or more, 80% or more, 90% or more, 95% or more, or 99% or more homology to SEQ ID NO:3 are also provided.

The sequence of the CDRs and intervening framework regions of the variable region of the light chain of the AB0024 antibody is (the sequenced of CDR1, CDR2, and CDR3 are underlined):

(SEQ ID NO: 4)
DIVMTQTPLSLSVTPGQPASISCRSSKSLLHSNGNTYLYWFLQKPGQS
PQFLIYRMSNLASGVPDRFSGSGSGTDFTLKISRVEAEDVGVYYCMQH
LEYPYTFGGGTKVEIK

Additional light chain variable region amino acid sequences having 75% or more, 80% or more, 90% or more, 95% or more, or 99% or more homology to SEQ ID NO:4 are also provided.

Additional anti-LOXL2 antibody sequences, including additional humanized variants of the variable regions, framework region amino acid sequences and the amino acid sequences of the complementarity-determining regions, are disclosed in co-owned United States Patent Application Publication No. US 2009/0053224 (Feb. 26, 2009), the disclosure of which is incorporated by reference in its entirety herein for the purpose of providing the amino acid sequences of various anti-LOXL2 antibodies.

LOXL2 as a Diagnostic Marker for Pulmonary Fibrotic Disorders

The increase in LOXL2 levels in pulmonary fibrotic tissue, disclosed herein, and the attendant decrease in LOXL2 levels that accompanies normalization of pulmonary architecture following treatment with LOXL2 inhibitors, also disclosed herein, indicate that LOXL2 level in pulmonary tissue can be used as a diagnostic marker for pulmonary fibrotic disorders. Accordingly, an increase in LOXL2 levels in lung tissue is indicative of onset or progression of a pulmonary fibrotic disorder.

Methods for measuring LOXL2 levels are known in the art and include assays for enzymatic activity, assays for LOXL2 protein and assays for LOXL2 mRNA. See, for example, United States patent application publications US 2006/0127402 (Jun. 15, 2006), 2009/0053224 (Feb. 26, 2009) and 2009/0104201 (Apr. 23, 2009), and Rodriguez et al. (2010) J. Biol. Chem. 285:20964-20974, the disclosures of which are incorporated by reference herein, in their entireties, for the purpose of describing assays for the detection, quantitation and inhibition of LOXL2.

LOXL2 as a Prognostic Marker for Pulmonary Fibrotic Disorders

Pulmonary fibrotic disorders can involve periods of relative stability punctuated by acute phases resulting in morbidity and/or death. Accordingly, good prognostic markers are required.

The inventors have determined that the overexpression of LOXL2 that is characteristic of pulmonary fibrotic disorders is reversed by treatment with LOXL2 inhibitors. As a result, LOXL2 levels in lung tissue can be used as a prognostic marker to assess the effectiveness of treatments for pulmonary fibrotic disorders, with decreases in LOXL2 levels being indicative of amelioration of symptoms and improved prognosis. Treatments can include steroid, antibiotic, or anti-neoplastic treatments, and/or treatments using LOXL2 inhibitors.

### EXAMPLES

### Example 1

### Model System

Bleomycin-induced pulmonary fibrosis in mice is a recognized, standard model system for IPF and other pulmonary fibrotic disorders. See, for example, Harrison and Lazo (1987) J. Pharmacol. Exp. Ther. 243:1185-1194; Walters and Kleeberger (2008) Current Protocols Pharmacol. 40:5.46.1-5.46.17. This system was used to study the effects of a LOXL2 inhibitor, in the form of an anti-LOXL2 antibody, on the course and outcome of lung fibrosis.

In brief, lung fibrosis was induced in male C57B/L6 mice by oropharyngeal administration of bleomycin. For bleomycin administration, animals were anaesthetized and suspended on their backs at an approximately 60° angle with a rubber band running under the upper incisors. The tongue was held with one arm of a set of padded forceps, thereby opening the airway. Bleomycin solution was introduced into the back of the oral cavity by pipette, and the tongue and mouth were held open until the liquid was no longer visible in the mouth.

Mice were also administered an anti-LOXL2 antibody (AB0023) either before (Prevention study: Example 2) or after (Treatment study: Example 3) bleomycin treatment. The AB0023 antibody is disclosed herein in the section entitled “Anti-LOXL2 Antibodies,” and has been described in co-owned US 2009/0053224 (Feb. 26, 2009), the disclosure of which is incorporated by reference herein for the purposes of describing anti-LOXL2 antibodies, their preparation and their properties.

### Example 2

### Prevention Study

In this study, 21 male C57BL/6 mice, at 7-8 weeks of age, were divided into three groups: Group 1 contained 5 animals and Groups 2 and 3 contained 8 animals each. Group 1 was a control group in which animals were treated with saline on Day 0 and twice weekly thereafter. Animals in Group 2 received 1 Unit/kg bleomycin on day 0. Four days and one day prior to bleomycin administration, animals in Group 2 also received injections of antibody diluent (PBS), and they received injections of antibody diluent twice weekly after administration of bleomycin. Animals in Group 3 received 1 Unit/kg bleomycin on day 0. Four days and one day prior to bleomycin administration, animals in Group 3 were injected with 15 mg/kg anti-LOXL2 antibody (AB0023), and they received injections of 15 mg/kg antibody twice weekly after administration of bleomycin. The study design is shown in Table 1.

Bleomycin sulfate (MP Biomedicals, Catalogue #19030, Lot 2373K) was dissolved in 0.9% saline and was administered oropharyngeally under anaesthesia to give a final concentration of 1 Unit per kilogram body weight. See Walters & Kleeberger, supra. AB0023 was administered by intraperitoneal injection of a 3 mg/ml stock solution in PBS to give a final concentration of 15 mg/kg. Vehicle (PBS) was administered by intraperitoneal injection.

TABLE 1
Prevention Study Design

| Group | n | Day −4 | Day −1 | Day 0 | 2x weekly | Day 14 |
|---|---|---|---|---|---|---|
| 1 | 5 | — | — | saline | — | sacrifice |
| 2 | 8 | vehicle | vehicle | 1 U/kg bleo | vehicle | sacrifice |
| 3 | 8 | Ab0023 | Ab0023 | 1 U/kg bleo | Ab0023 | sacrifice |

On Day 14, the study was terminated and all animals were sacrificed for analysis. Blood was collected by cardiac puncture and used for preparation of serum. The lungs were dissected out and weighed. Lungs from half of the animals in each group were used for collection of bronchioalveolar lavage fluid by perfusion with Hanks' Balanced Salt Solution. Lavage fluid was centrifuged and the supernatant removed and frozen. Cells in the pellet were resuspended in 2 ml of 1× Pharmalyse Buffer (BD Biosciences, San Jose, Calif.) to lyse erythrocytes. Lysis was terminated by addition of PBS+2% bovine serum albumin and the cells were centrifuged. Leukocytes in the pellet were identified by Trypan Blue exclusion and counted using a hemocytometer.

After lavage, these lungs were fixed in 10% neutral buffered formalin. Lungs from the remaining animals were snap-frozen in liquid N₂ and stored at −80° C. for histopathology and protein determination.

Immunohistochemistry (IHC) and Immunofluorescence (IF)

All solutions and reagents were obtained from Biocare Medical (Concord, Calif.), unless otherwise noted, and all procedures were conducted at room temperature. Sections (5 μm) were cut from either formalin-fixed or fresh frozen lung tissue and stained with either hematoxylin and eosin (H&E) or Sirius Red.

For IHC or IF on formalin-fixed tissue sections, antigen retrieval was conducted in a decloaking chamber at 90° C. for 45 min. Primary antibodies to collagen I, α-smooth muscle actin, transforming growth factor β-1 (TGF (3-1), endothelin-1 (ET-1), CD45 and stromal-derived factor-1α (SDF-1α/CXCL12) were obtained from AbCam (Cambridge, Mass.) and used at a concentration of 1-10 μg/ml.

Fresh frozen tissue was fixed with 4% paraformaldehyde, and IHC was performed using a primary anti-LOXL2 polyclonal antibody (Arresto Biosciences, Inc., Palo Alto, Calif.).

Prior to contact with primary antibody, sections were treated with Peroxidazed-1 (Biocare Medical, Concord, Calif.) to block endogenous peroxidase activities and with Background Sniper (Biocare Medical, Concord, Calif.).

The procedure for IHC was as follows. Primary antibody was added to the slide and incubated for 30 min. After washing, a horseradish peroxidase (HRP)-conjugated secondary antibody, (MACH 2, Biocare Medical, Concord, Calif.) was added and incubation was conducted for 30 min. After washing off secondary antibody the slide was incubated with diaminobenzidine (DAB) chromogen for 1 min, then counterstained with hematoxylin.

For IF on sections of formalin fixed tissue, a solution of primary antibody (rat anti-CD45 or goat anti-collagen I) was added to the slide and incubated for one hour. After washing, a mixture of Alexa Fluor 488 (green) goat anti-rabbit and Alexa Fluor 546 (red) goat anti-rat secondary antibodies (both from Invitrogen, Carlsbad, Calif.) was added and incubation was conducted for one hour. Slides were counterstained with DAPI, mounted, and viewed in a fluorescence microscope. For visualization of Alexa Fluor 488 (green, indicating collagen) an excitation wavelength of 495 nm and an emission wavelength of 519 nm was used. For visualization of Alexa Fluor 546 (red, indicating CD45) an excitation wavelength of 556 nm and an emission wavelength of 573 nm was used.

For each of the three treatment groups, 3-4 fields from different lungs were tested for each antigen. Signal area per field was quantitated using MetaMorph imaging software (Molecular Devices, Downingtown, Pa.).

ELISA Assay for pSMAD2

Lung tissue was homogenized in Cell Lysis Buffer (Cell Signaling Technology, Inc., Danvers, Mass.) containing 1 mM PMSF, and the homogenate was used in an ELISA assay for phosphorylated SMAD2 (p-SMAD2).

Results

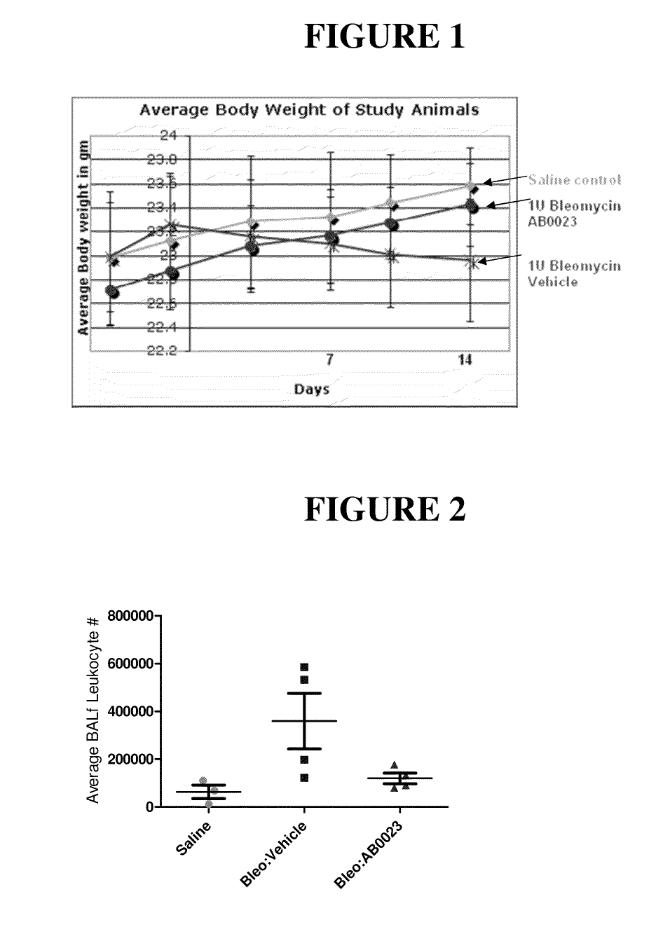

Animals treated with bleomycin exhibited limited weight gain or a small degree of weight loss throughout the study (e.g., group 2). However, bleomycin-treated animals that were also treated with AB0023 (e.g., Group 3) showed steady weight gain (FIG. 1). As expected, the saline treated control animals (Group 1) also exhibited steady weight gain throughout the study.

Total leukocyte numbers in BAL fluid were higher in bleomycin-treated animals (Group 2) compared to a saline control group (Group 1). See FIG. 2. In additional experiments, a correlation was observed between higher concentrations of bleomycin and higher total leukocyte numbers in BAL fluid. Treatment with Ab0023 resulted in a reduction of leukocyte numbers in the BAL of bleomycin-treated animals to levels similar to those observed in the saline controls (FIG. 2). Similar results were obtained in a second study (p=0.032).

Treatment of mice with 1 Unit/kg bleomycin (Group 2) evoked a robust fibrotic response, as evidenced by increased levels of crosslinked collagen and proliferation of α-SMA-positive cells (“activated fibroblasts” or “fibrocytes”). Lung architecture was also distorted, as evidenced by alveolar thickening and proliferation of pneumocytes (primarily Type II pneumocytes).

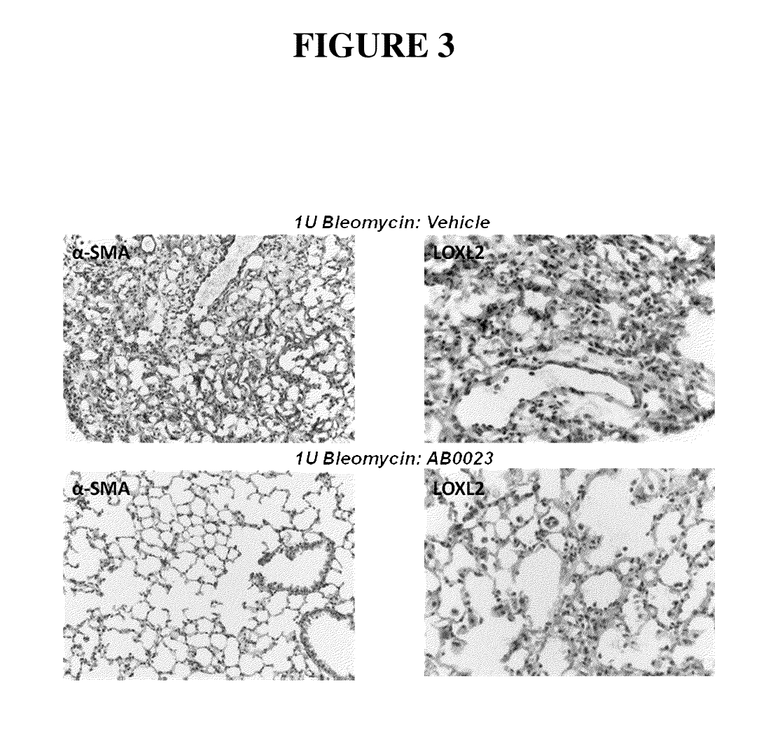

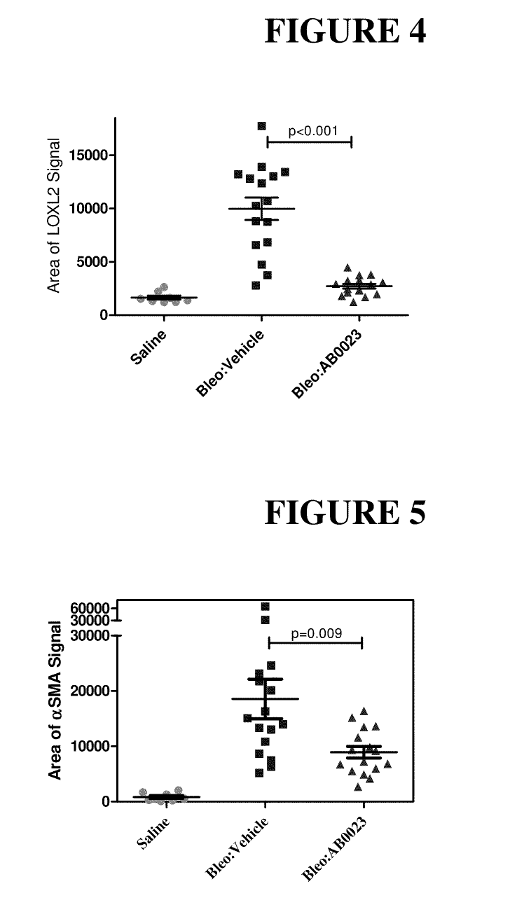

Analysis of lungs from study animals revealed that LOXL2 expression was induced in lung epithelial cells (Type I and Type II pneumoyctes) and in infiltrating fibroblasts as a consequence of bleomycin treatment (FIG. 3, upper right panel; FIG. 4). Cells positive for α-smooth muscle actin (α-SMA), used to identify activated fibroblasts or myofibroblasts, were also prevalent in lungs from bleomycin-treated mice (FIG. 3, upper left panel; FIG. 5). Animals exposed to bleomycin, that were also treated with Ab0023, exhibited significantly lower levels of both LOXL2 and α-SMA-positive cells (FIG. 3, lower panels, FIG. 4, FIG. 5).

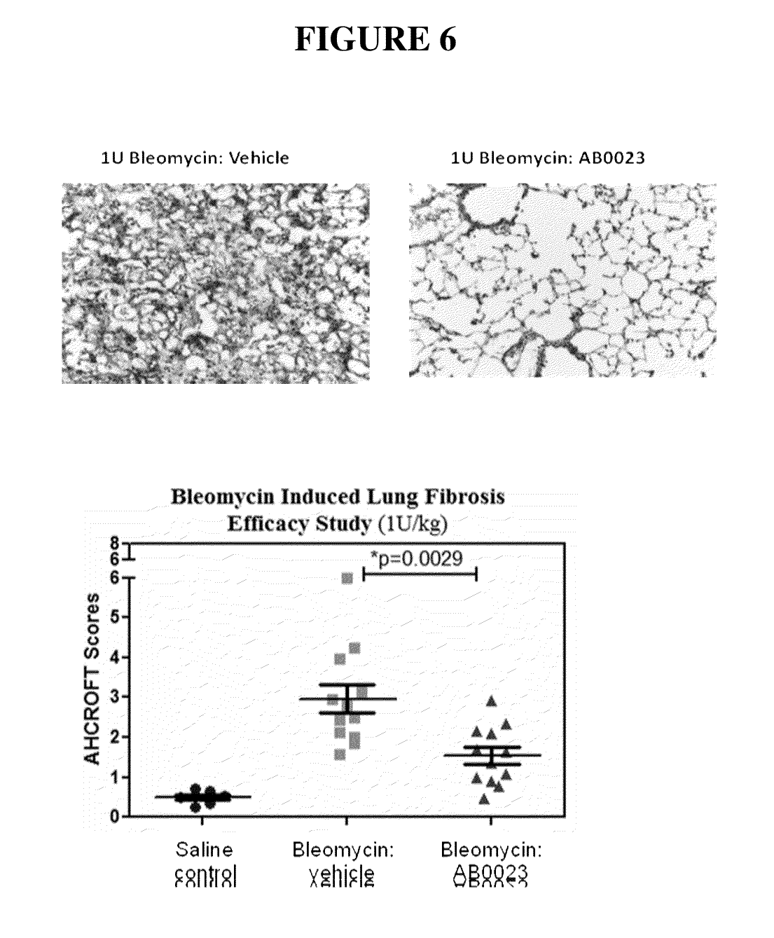

Lung damage was assessed morphologically using Ashcroft scoring guidelines. See Ashcroft et al. (1988) J. Clin. Pathol. 41:467-470. Analyses were conducted by three different individuals, blinded to study group identification. Lungs from animals in the saline control group had Ashcroft scores of <1. The average Ashcroft score from bleomycin-vehicle treated animals was 3, and this score was significantly reduced by treatment with AB0023 (p=0.0029, FIG. 6). Thus, lung architecture was also restored by treatment with anti-LOXL2 antibody.

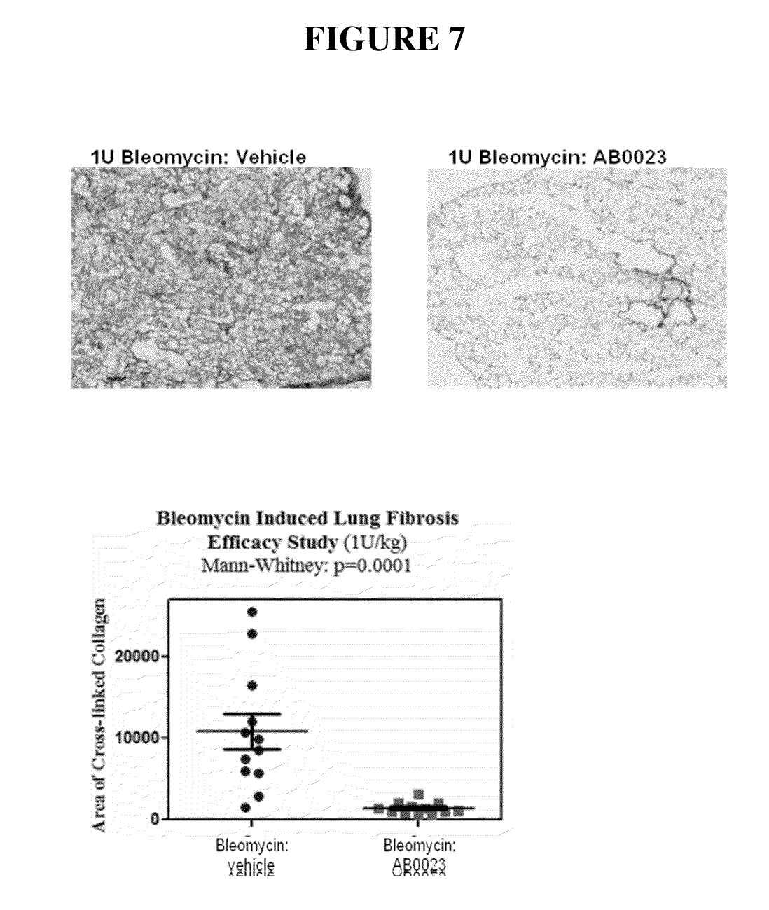

An independent quantitative assessment of fibrosis was made using Sirius Red staining to detect cross-linked fibrillar collagen. Lungs from bleomycin-treated animals contained large amounts of cross-linked collagen (FIG. 7, top left); while administration of anti-LOXL2 antibody to bleomycin-treated animals greatly reduced the amount of cross-linked collagen (indicative of lung fibrosis) that resulted from exposure to bleomycin (FIG. 7, top right). Quantitation of signal area, viewed under polarized light, indicated that the reduction was statistically significant, (p=0.0001, FIG. 7, bottom).

TGFβ-1 and SDF-1α have been identified as disease drivers in both human fibrotic lung disease and in the bleomycin-induced fibrosis model. Stromal-derived factor-1 (SDF-1) is a chemokine, elaborated primarily by neutrophils and macrophages, whose receptor, CXCR4, is found on a small population of bone marrow stem cells. SDF-1 exists in two forms, produced by alternative splicing: SDF-1α and SDF-β. In the pathology of IPF, SDF-1α is believed to mediate recruitment of these CXCR4⁺ stem cells to the lung, where they differentiate into fibrocytes and elaborate collagen, contributing to fibrotic damage. See, e.g., Xu, et al. (2007) Am. J. Resp. Cell. Mol. Biol. 37:291-299. For the role of TGF-β1 in IPF, see Noble (2008) Eur. Respir. Rev. 17:123-129.

Because of the role of these proteins in the pathology of IPF, the effects of anti-LOXL2 AB0023 on their expression in fibrotic lungs was assessed using immunohistochemistry.

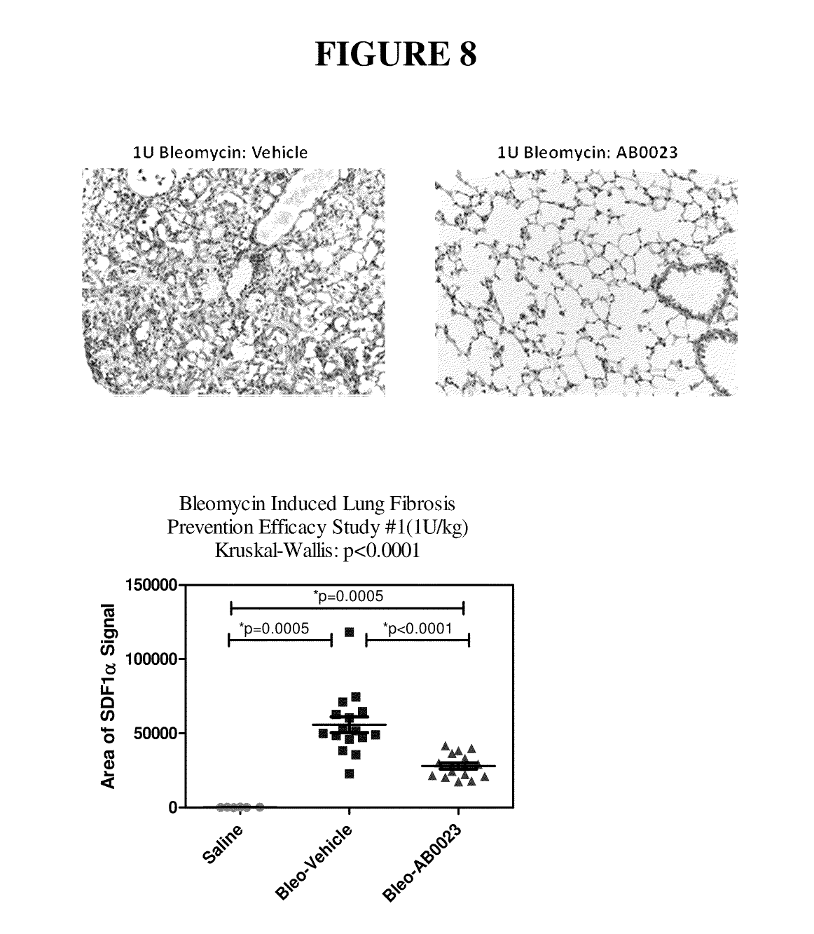

SDF-1α levels were substantially increased compared to saline controls in the lungs of animals treated with bleomycin (FIG. 8, top left), with expression by type II pneumocytes, potential fibrocytes and possibly other cell types. Treatment with AB0023 significantly reduced SDF-1α expression resulting from bleomycin exposure (FIG. 8, top right). Quantitation of signal area indicated that the reduction was statistically significant (p<0.0001, FIG. 8, bottom).

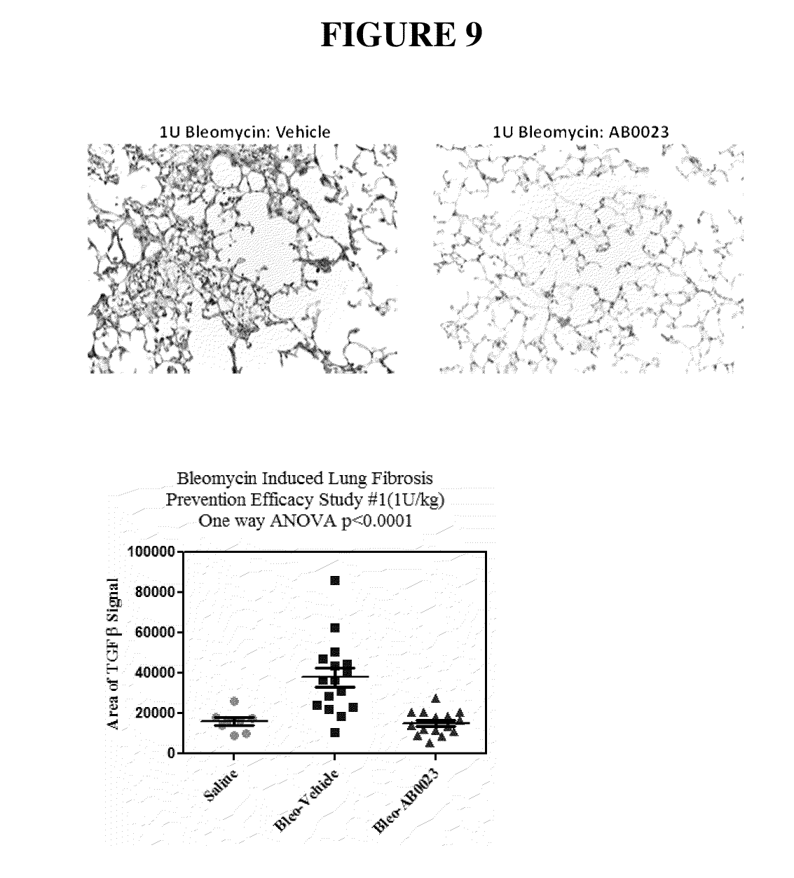

Bleomycin treatment resulted in the expression of TGFβ-1 by a variety of cell types in the lung, including macrophages, type II pneumocytes, myofibroblasts and possibly fibrocytes (FIG. 9, top left). TGFβ-1 levels were significantly reduced (FIG. 9, top right) in the lungs of animals treated with AB0023 (p<0.0001, FIG. 9, bottom).

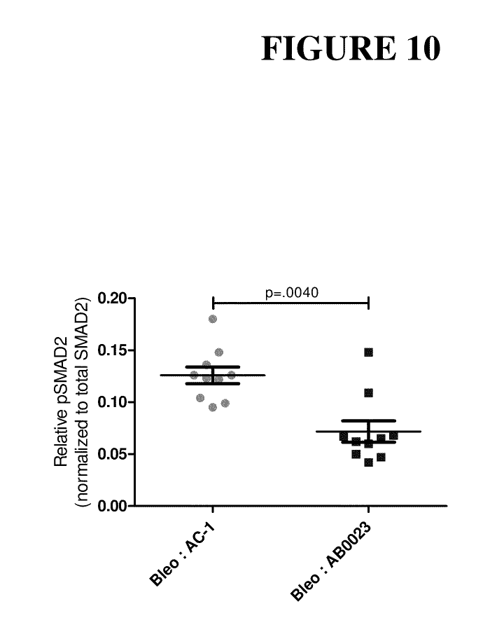

In a separate study, levels of phosphorylated SMAD2 (p-SMAD2), a marker of the activation of the TGFβ-1 signaling pathway, were determined. In mice that had been treated with bleomycin to induce lung fibrosis, a tissue-based ELISA assay revealed a decrease in the phosphorylation of SMAD2 in mice treated with the anti-LOXL2 antibody, compared to mice treated with a control antibody (FIG. 10).

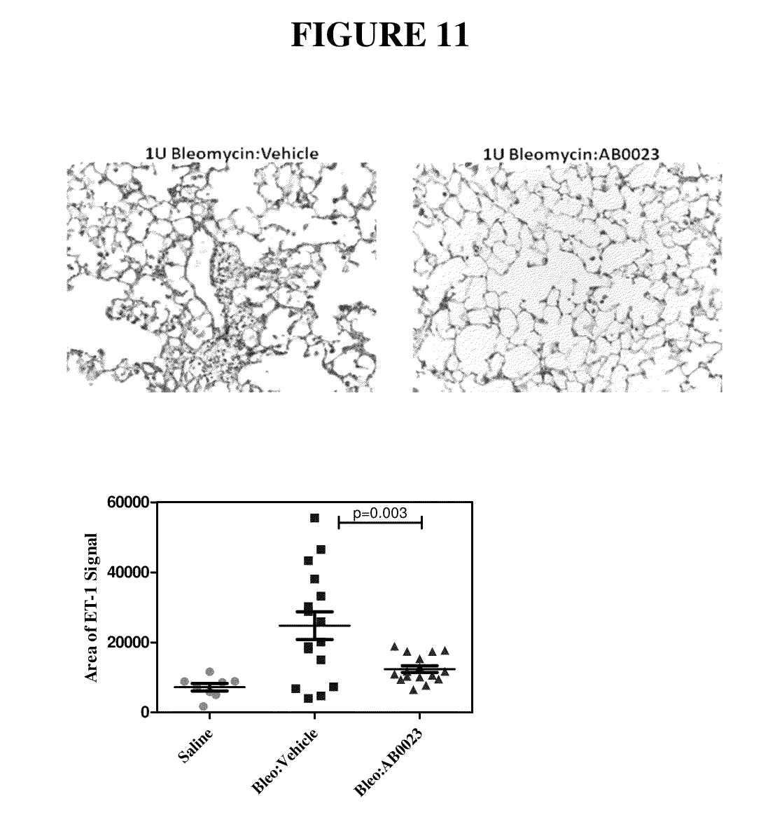

Expression of endothelin-1 (ET-1) is induced by TGFβ-1, and endothelin-1 collaborates with TGFβ-1 in the pathogenesis of lung fibrosis. Analysis by immunohistochemistry indicated that the pattern of ET-1 expression was very similar to that of TGFβ-1 in bleomycin treated animals (FIG. 11, top left) and a significant decrease of ET-1 was also observed upon AB0023 treatment (FIG. 11, top right). Quantitation of signal area indicated that the reduction was statistically significant (p=0.005, FIG. 11 bottom).

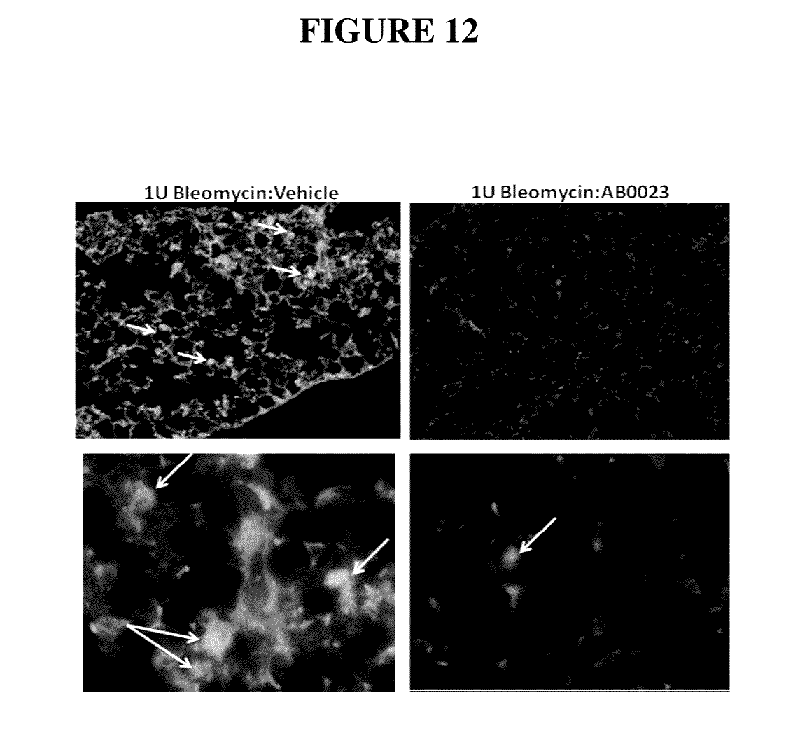

One of the sources of the collagen-producing cells in fibrotic lungs appears to be derived from a CD45-positive hematopoietic stem cell. These precursor cells (“fibrocytes”) can be detected in tissue sections by co-localization of reactivity for collagen and CD45. When sections from lungs of the animals in Groups 2 and 3 were examined by immunofluorescence for type I collagen and CD45, lungs from bleomycin-treated animals (Group 2) were found to possess many fibrocytes (FIG. 12, left panels). Fewer fibrocytes were found in lungs from the Group 3 animals that had received both bleomycin and the anti-LOXL2 antibody (FIG. 12, right panels).

Conclusions

Treatment with an anti-LOXL2 antibody improved general health (as evidenced by increased body weight), normalized leukocyte count in BAL fluid, reduced fibrosis, reduced alveolar thickening, improved lung architecture, reduced fibrocyte numbers, reduced the number of CD45⁺/Collagen I⁺ fibrocyte precursors, and improved Ashcroft score in mice with bleomycin-induced pulmonary fibrosis. Moreover, levels of the following proteins (all of which are markers of fibrotic tissue) were reduced by anti-LOXL2 treatment: LOXL2, transforming growth factor β-1 (TGFβ-1), endothelin-1, p-SMAD2 and stromal-derived factor-1α (SDF-1α/CXCL12). These results demonstrate the effectiveness of LOXL2 inhibitors, particularly anti-LOXL2 antibodies, in reducing severity and reversing symptoms of pulmonary fibrosis.

### Example 3

### Treatment Study

In this study, mice were administered bleomycin and allowed to develop pulmonary fibrosis, then treated with either an anti-LOXL2 antibody (AB0023) or a control antibody (AC-1).

Study Design

C57BL/6 mice, 7-8 weeks of age, were divided into three groups. Group 1 (controls) consisted of five animals, while Groups 2 and 3 consisted of 8 animals each. On day 0, animals in Groups 2 and 3 were exposed to bleomycin as described in Example 2, except that the dose was 2.5 Units/kg. Control animals in Group 1 were administered an equal volume of saline, using the same methods. No further treatment was administered to the animals in Group 1, and they were sacrificed on Day 14. On day 7, animals in Group 2 received 15 mg/kg of AC-1 antibody (control) and animals in Group 3 received 15 mg/kg of the anti-LOXL2 antibody AB0023. Administration of antibody was by intraperitoneal (IP) injection. Administration of antibodies to animals in Groups 2 and 3 was continued twice weekly thereafter, using the same antibodies, concentration and route of administration. On Day 22, animals in Groups 2 and 3 were sacrificed for analysis. The study design is summarized in Table 2.

TABLE 2
Treatment Study Design

| Group | n | Day 0 | Day 7 | 2x weekly | Day 14 | Day 22 |
|---|---|---|---|---|---|---|
| 1 | 5 | saline | | | sacrifice | — |
| 2 | 8 | 2.5 U/kg | 15 mg/kg | 15 mg/kg | — | sacrifice |
| | | bleomycin | AC-1 | AC-1 | | |
| 3 | 8 | 2.5 U/kg | 15 mg/kg | 15 mg/kg | — | sacrifice |
| | | bleomycin | AB0023 | AB0023 | | |

Analysis

All animals were weighed prior to bleomycin exposure and then twice weekly until termination of the study.

At the time of harvest, blood was collected by terminal cardiac bleed and serum was prepared. Lungs from some of the animals were dissected out and weighed. Lungs from the remaining animals were snap frozen in liquid nitrogen and stored at −80° C. for histopathology or were fixed using 10% neutral buffered formalin.

The solutions used for immunohistochemical (IHC) analyses were obtained from Biocare Medical (Concord, Calif.). All procedures were performed at room temperature. Sections were generated and stained with Sirius red, anti-LOXL2 polyclonal antibody (Arresto Biosciences, Palo Alto, Calif.), and anti-αSMA antibodies (1:250; Abcam, Cambridge, Mass.). Antigen retrieval was performed on five μm sections of formalin fixed tissue, endogenous peroxidase was blocked with Peroxidazed-1 (Biocare Medical), and background was blocked with Background Sniper (Biocare Medical). Sections were stained with primary antibodies for 30 minutes, incubated with secondary antibody (MACH 2 HRP-conjugated anti-rabbit, Biocare Medical, Concord, Calif.) for 30 minutes; incubated with DAB for 1 min and counterstained with hematoxylin. Four fields from 5 randomly-chosen lungs were stained for each treatment. Area occupied by signal in the lung sections was quantified using MetaMorph Imaging Software (Molecular Devices, Downingtown, Pa.), which measures the area of DAB staining in the section.

Results

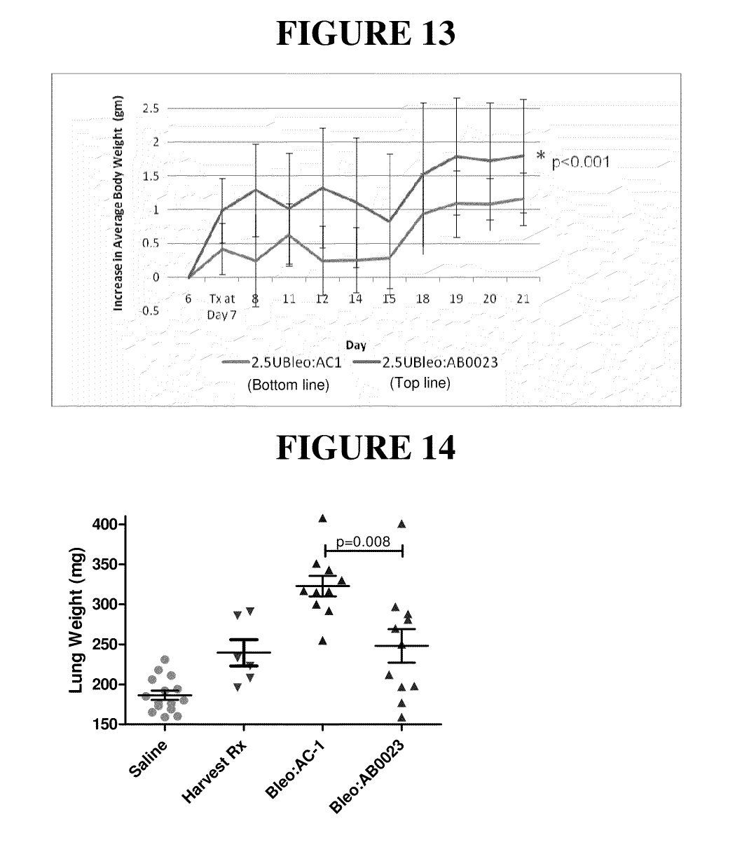

Body Weight: On day 22, AB0023-treated animals (post bleomycin exposure, group 3) had gained an average of 1.34 grams since the start of antibody treatment on day 7, whereas AC-1 treated animals (group 2) had gained an average of only 0.64 grams within the same time frame (FIG. 13). As expected, the saline-treated control animals (group 1) also showed steady weight gain throughout the study.

Inasmuch as decreased body weight is a symptom of IPF and other pulmonary fibrotic disorders, these results show that treatment with a LOXL2 inhibitor reduces symptoms of pulmonary fibrotic disorders such as IPF.

Lung weight: Seven bleomycin-treated animals, chosen from Groups 2 and 3, were sacrificed within 24-48 hour of the administration of antibodies on Day 7. These animals were denoted the “Harvest Rx” sample. The lungs from these animals had an average weight of 239.5 mg. By comparison, lungs from control animals (Group 1, saline-treated) sacrificed at Day 22 had an average weight of 186.4 mg. At the end of the treatment period (i.e., at Day 22), average lung weight from bleomycin-treated animals that had received injections of the AC-1 control antibody (group 2) had increased from the Harvest Rx value of 239.5 mg to an average of 322.7 mg, whereas the average weight of lungs from bleomycin-treated animals that had received injections of the AB0023 anti-LOXL2 antibody (group 3) increased only slightly above the Harvest Rx value, to 248.2 mg. These data are shown in FIG. 14. Thus, the LOXL2 inhibitor prevented the increase in lung weight caused by bleomycin treatment.

Inasmuch as an increase in lung weight is a symptom of IPF and other pulmonary fibrotic disorders, these results show that treatment with a LOXL2 inhibitor reduces symptoms of pulmonary fibrotic disorders such as IPF.

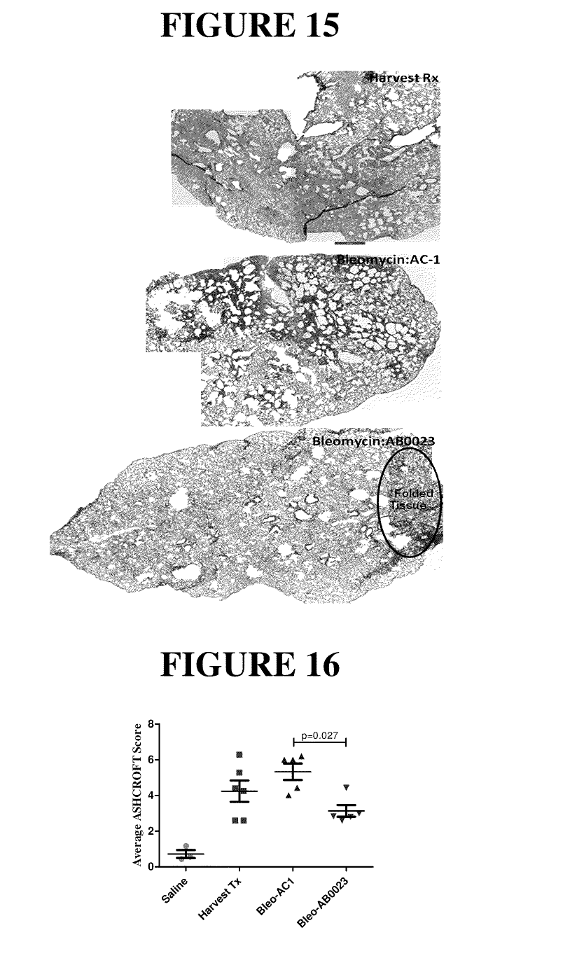

Lung architecture: Analysis by immunohistochemistry revealed that a robust fibrotic response had been evoked in the lungs of bleomycin-treated animals (FIG. 15). Lung damage included alveolar thickening, presence of fibrotic foci and honeycomb lung. Treatment with the anti-LOXL2 antibody reduced and reversed this damage, restoring a closer-to-normal lung architecture to the bleomycin-treated animals (e.g., FIG. 15, bottom panel).

Ashcroft Score: Lung damage was also assessed using Ashcroft scoring guidelines (Ashcroft et al., supra). See FIG. 16. Assessment was conducted by individuals that were blinded to study group identification. Lungs from animals in the saline control group (group 1) had Ashcroft scores <1. At the beginning of treatment (Harvest Rx), lungs from bleomycin-treated animals had an average Ashcroft score of 4.23. On day 22, lungs from bleomycin-treated animals that had received injections of AC-1 (group 2) exhibited evidence of severe disease with multiple instances of patchy honeycomb lung (FIG. 15, middle panel), and had an Ashcroft score of 5.33. Lungs from bleomycin-treated animals that had received injections of AB0023 (group 3) had an average Ashcroft score of 3.13 (FIG. 16). Thus, not only did treatment with AB0023 significantly inhibit the progression of fibrosis, compared to AC-1 treatment (p<0.027, FIG. 16), it also reversed the damage characteristic of the lungs isolated from animals near the start of treatment. The histologic appearance of the lungs in AB0023-treated animals at day 22 (FIG. 15, bottom panel) was similar to that of saline-treated animals, aside from a slight increase in the number of type II pneumocytes; the Ashcroft scores reflects this finding.

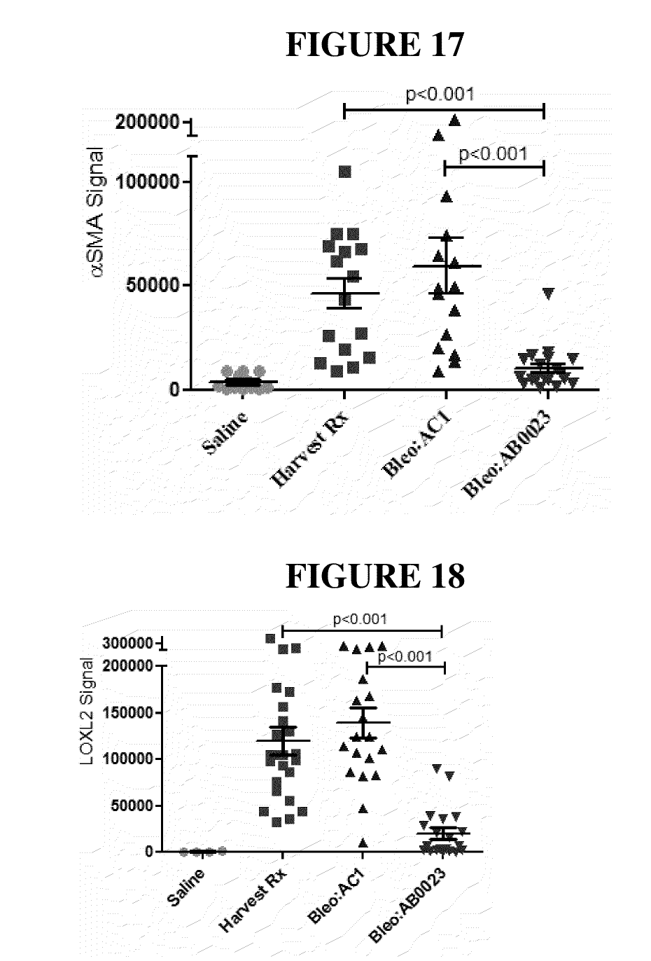

Immunohistochemistry: Lungs from control and antibody-treated animals were tested for alpha-smooth muscle actin (α-SMA) immunoreactivity (a characteristic of activated fibroblasts); and for LOXL2 immunoreactivity. These analyses, carried out on lungs harvested on day 22, showed statistically significant reductions in α-SMA levels (FIG. 17), and in LOXL2 levels (FIG. 18), in lungs from AB0023-treated animals (group 3), relative to lungs from AC-1 treated animals (group 2). Furthermore, Harvest Rx samples from bleomycin-treated animals showed extensive fibroblast activation (evidenced by an increase in α-SMA-positive cells compared to normal lung), that was reversed in the Day 22 samples from AB0023-treated animals (FIG. 17).

The IHC analyses also revealed that LOXL2 expression was coincident with areas of fibroblastic foci in lungs harvested at the beginning of treatment (Harvest Rx sample) and in AC-1 treated lungs. Furthermore, Harvest Rx samples from bleomycin-treated animals showed extensive collagen deposition (evidenced by an increase in LOXL2 signal compared to saline-treated controls), that was reversed in the Day 22 samples from AB0023-treated animals (FIG. 18).

These data demonstrate that LOXL2 plays an important role in promoting and sustaining lung fibrosis and that LOXL2 inhibitors (such as, for example, anti-LOXL2 antibodies) not only reduce, but also reverse, lung injury through, inter alia, inhibition of fibroblast activation and collagen deposition.

Epithelial morphology: Analyses of H&E-stained sections under high magnification showed that administration of AB0023 to bleomycin-treated animals reduced fibrosis in the alveolar walls and reversed the expansion of pneumocytes that had accompanied bleomycin-induced fibrosis.

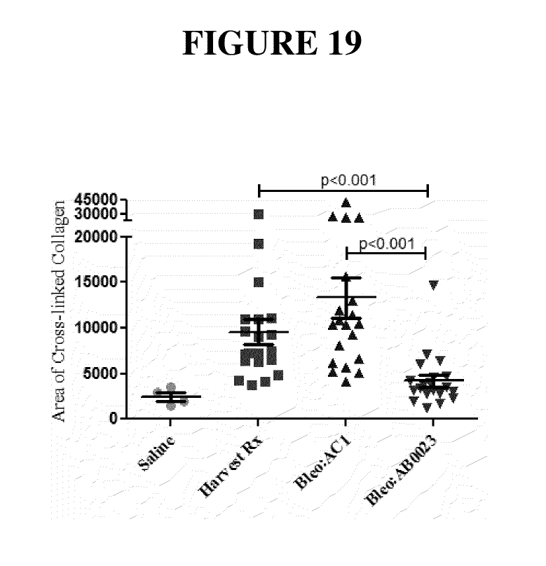

Collagen levels: Lungs from control and antibody-treated animals were stained with Sirius Red, and the stained sections were analyzed under polarized light. Under these conditions, degree of staining reflects levels of fibrillar cross-linked collagen. The results of these analyses showed an increase in levels of fibrillar cross-linked collagen in bleomycin-treated animals shortly after initiation of antibody treatment (Harvest Rx sample), and a statistically significant reduction in levels of cross-linked collagen (i.e., reversal of fibrosis) in sections of lung from bleomycin-treated animals that had received injections of AB0023, compared to Bleomycin-treated animals that had received injections of AC-1 (FIG. 19).

Staining with Masson's Trichrome (another collagen-specific reagent) confirmed the reduction in collagen deposition in AB0023-treated fibrotic lungs, compared to AC-1-treated fibrotic lungs, providing further support for the reversal of fibrotic damage following AB0023 treatment.

Conclusions

Treatment with a LOXL2 inhibitor (i.e., the anti-LOXL2 antibody AB0023) in a bleomycin-induced model of established lung fibrosis resulted in a significant reduction in fibrosis and in the number of activated fibroblasts, and normalization of lung architecture, lung weight, and body weight. In addition, the reduction of activated fibroblasts and the reduction in levels of LOXL2 itself, that accompanied AB0023 treatment, promoted reversal of fibrotic symptoms, recovery and protection of lung epithelia.

What is claimed is:

1. A method for the prevention of a pulmonary fibrotic disorder in a subject, the method comprising administering to the subject an inhibitor of the activity of the lysyl oxidase-related-2 protein (LOXL2).

2. The method of claim 1, wherein the pulmonary fibrotic disorder is selected from the group consisting of interstitial pneumonia, acute respiratory distress syndrome (ARDS) and idiopathic pulmonary fibrosis (IPF).

3. The method of claim 1, wherein the inhibitor is an antibody to LOXL2.

4. The method of claim 3, wherein the antibody comprises heavy chain sequences as set forth in SEQ ID NO:1 and light chain sequences as set forth in SEQ ID NO:2.

5. The method of claim 3, wherein the antibody is a humanized antibody.

6. The method of claim 5, wherein the antibody comprises heavy chain sequences as set forth in SEQ ID NO:3 and light chain sequences as set forth in SEQ ID NO:4.

7. A method for the treatment of a pulmonary fibrotic disorder in a subject, the method comprising administering to the subject an inhibitor of the activity of the lysyl oxidase-related-2 protein (LOXL2).

8. The method of claim 7, wherein the pulmonary fibrotic disorder is selected from the group consisting of interstitial pneumonia, acute respiratory distress syndrome (ARDS) and idiopathic pulmonary fibrosis (IPF).

9. The method of claim 7, wherein the inhibitor is an antibody to LOXL2.

10. The method of claim 9, wherein the antibody comprises heavy chain sequences as set forth in SEQ ID NO:1 and light chain sequences as set forth in SEQ ID NO:2.

11. The method of claim 9, wherein the antibody is a humanized antibody.

12. The method of claim 11, wherein the antibody comprises heavy chain sequences as set forth in SEQ ID NO:3 and light chain sequences as set forth in SEQ ID NO:4.

13. A method for reversing the symptoms of a pulmonary fibrotic disorder in a subject, the method comprising administering to the subject an inhibitor of the activity of the lysyl oxidase-related-2 protein (LOXL2).

14. The method of claim 13, wherein the pulmonary fibrotic disorder is selected from the group consisting of interstitial pneumonia, acute respiratory distress syndrome (ARDS) and idiopathic pulmonary fibrosis (IPF).

15. The method of claim 13, wherein the inhibitor is an antibody to LOXL2.

16. The method of claim 15, wherein the antibody comprises heavy chain sequences as set forth in SEQ ID NO:1 and light chain sequences as set forth in SEQ ID NO:2.

17. The method of claim 15, wherein the antibody is a humanized antibody.

18. The method of claim 17, wherein the antibody comprises heavy chain sequences as set forth in SEQ ID NO:3 and light chain sequences as set forth in SEQ ID NO:4.

19. The method of claim 13, wherein the symptom is selected from the group consisting of decreased body weight, increased lung weight, fibrosis, lung architecture, increased Ashcroft score, increased pulmonary collagen levels, and increased number of CD45⁺/collagen⁺ cells.

20. The method of claim 13, wherein the symptom is an increased level of one or more molecules selected from the group consisting of LOXL2, α-smooth muscle actin (α-SMA), transforming growth factor β-1 (TGFβ-1), stromal derived factor-1α (SDF-1α), endothelin-1 (ET-1) and phosphorylated SMAD2.

21. The method of claim 13, wherein the symptom is increased leukocyte number in bronchioalveolar lavage (BAL) fluid.

22. A pharmaceutical composition for the prevention or treatment of a pulmonary fibrotic disorder, or for reversing the symptoms of a pulmonary fibrotic disorder in a subject, wherein the composition comprises an inhibitor of the activity of the lysyl oxidase-related-2 protein (LOXL2) and a pharmaceutically acceptable excipient.

23. The composition of claim 22, wherein the pulmonary fibrotic disorder is selected from the group consisting of interstitial pneumonia, acute respiratory distress syndrome (ARDS) and idiopathic pulmonary fibrosis (IPF).

24. The composition of claim 22, wherein the inhibitor is an antibody to LOXL2.

25. The composition of claim 24, wherein the antibody comprises heavy chain sequences as set forth in SEQ ID NO:1 and light chain sequences as set forth in SEQ ID NO:2.

26. The composition of claim 24, wherein the antibody is a humanized antibody.

27. The composition of claim 26, wherein the antibody comprises heavy chain sequences as set forth in SEQ ID NO:3 and light chain sequences as set forth in SEQ ID NO:4.

28. The composition of claim 22, wherein the symptom is selected from the group consisting of decreased body weight, increased lung weight, fibrosis, lung architecture, increased Ashcroft score, increased pulmonary collagen levels, and increased number of CD45⁺/collagen⁺ cells.

29. The composition of claim 22, wherein the symptom is an increased level of one or more molecules selected from the group consisting of LOXL2, α-smooth muscle actin (α-SMA), transforming growth factor β-1 (TGFβ-1), stromal derived factor-1α (SDF-1α), endothelin-1 (ET-1) and phosphorylated SMAD2.

30. The composition of claim 22, wherein the symptom is increased leukocyte number in bronchioalveolar lavage (BAL) fluid.

31. A method for diagnosing a pulmonary fibrotic disorder in a subject, the method comprising:
(a) obtaining a sample of pulmonary tissue from the subject; and
(b) determining the levels of LOXL2 in the sample;
wherein an increased level of LOXL2 in the sample, compared to a control sample, indicates the existence of a pulmonary fibrotic disorder.

32. The method of claim 31, wherein the pulmonary fibrotic disorder is selected from the group consisting of interstitial pneumonia, acute respiratory distress syndrome (ARDS) and idiopathic pulmonary fibrosis (IPF).

33. The method of claim 31, wherein the levels of LOXL2 in the sample are determined by contacting the sample with an antibody to LOXL2, so as to allow the formation of a complex between the antibody and the LOXL2 in the sample, and measuring the amount of complex that is formed.

34. The method of claim 33, wherein the antibody comprises heavy chain sequences as set forth in SEQ ID NO:1 and light chain sequences as set forth in SEQ ID NO:2.

35. The method of claim 33, wherein the antibody is a humanized antibody.

36. The method of claim 35, wherein the antibody comprises heavy chain sequences as set forth in SEQ ID NO:3 and light chain sequences as set forth in SEQ ID NO:4.

37. A method for monitoring a subject's response to a therapy for treating a pulmonary fibrotic disorder, the method comprising:
(a) obtaining a sample of pulmonary tissue from the subject; and
(b) determining the levels of LOXL2 in the sample;
wherein a decreased level of LOXL2 in the sample, compared to a control sample, indicates an amelioration of the pulmonary fibrotic disorder.

38. The method of claim 37, wherein the pulmonary fibrotic disorder is selected from the group consisting of interstitial pneumonia, acute respiratory distress syndrome (ARDS) and idiopathic pulmonary fibrosis (IPF).

39. The method of claim 37, wherein the levels of LOXL2 in the sample are determined by contacting the sample with an antibody to LOXL2, so as to allow the formation of a complex between the antibody and the LOXL2 in the sample, and measuring the amount of complex that is formed.

40. The method of claim 39, wherein the antibody comprises heavy chain sequences as set forth in SEQ ID NO:1 and light chain sequences as set forth in SEQ ID NO:2.

41. The method of claim 39, wherein the antibody is a humanized antibody.

42. The method of claim 41, wherein the antibody comprises heavy chain sequences as set forth in SEQ ID NO:3 and light chain sequences as set forth in SEQ ID NO:4.

43. The method of claim 37, wherein the treatment comprises administering, to the subject, an inhibitor of LOXL2.

44. The method of claim 43, wherein the inhibitor is an antibody.

45. The method of claim 44, wherein the inhibitor comprises heavy chain sequences as set forth in SEQ ID NO:1 and light chain sequences as set forth in SEQ ID NO:2.

46. The method of claim 44, wherein the inhibitor is a humanized antibody.

47. The method of claim 46, wherein the inhibitor comprises heavy chain sequences as set forth in SEQ ID NO:3 and light chain sequences as set forth in SEQ ID NO:4.
